(19) United States
(12) Patent Application Publication
(10) Pub. No.: US 2026/0152569 A1
(43) Pub. Date: Jun. 4, 2026

(54) ANTI-GPC1 MONOCLONAL ANTIBODY, THERAPEUTIC AND DIAGNOSTIC USES THEREOF

(71) Applicants: CENTRO DI RIFERIMENTO ONCOLOGICO DI AVIANO, Aviano (Pordenone) (IT); UNIVERSITA' DEGLI STUDI DI TRIESTE, Trieste (IT)

(72) Inventors: Giuseppe TOFFOLI, Sacile (Pordenone) (IT); Michele DAL BO, Aviano (Pordenone) (IT); Paolo MACOR, Fossalta di Portogruaro (Venezia) (IT); Davide BUSATO, Aviano (Pordenone) (IT); Sara CAPOLLA, Pasian di Prato (Udine) (IT); Federica DI CINTIO, Pescara (IT)

(21) Appl. No.: 19/122,386

(22) PCT Filed: Oct. 18, 2023
(86) PCT No.: PCT/EP2023/079012

§ 371 Date: Apr. 18, 2025

(30) Foreign Application Priority Data
Oct. 19, 2022 (IT) 102022000021546

(51) Int. Cl. C07K 16/30; A61K 39/00; A61K 47/68; A61K 47/69; A61P 35/00; G01N 33/575

(52) U.S. Cl. CPC C07K 16/303; A61K 47/6809; A61K 47/6859; A61K 47/6925; A61P 35/00; G01N 33/5758; A61K 2039/505; C07K 2317/734

(57) ABSTRACT

A monoclonal antibody of IgM isotype which specifically binds to the antigen glypican-1 (GPC1) in vitro, ex vivo and in vivo, is able to induce complement dependent cytotoxicity and reduce GPC1-expressing tumor masses in vivo. A nucleic acid construct and a transformed host cell are suitable for use in a method of recombinantly producing the anti-GPC1 monoclonal antibody and in diagnostic and therapeutic methods.

### TECHNICAL FIELD

The present invention pertains to a monoclonal antibody suitable for the diagnosis and therapy of tumor diseases, more in particular tumors that express the tumor-associated antigen Glypican-1 (GPC1). The invention also pertains to a method of manufacturing the monoclonal antibody and to the diagnostic and therapeutic uses thereof.

### BACKGROUND ART

GPC1 represents a molecular target that offers great opportunities for the treatment of various solid tumors in which it is specifically expressed at high levels.

GPC1 is a proteoglycan consisting of 558 amino acids expressed on the cell surface. It consists of an N-terminal region and a C-terminal region terminated with a glycosylphosphatidylinositol anchor (GPI anchor), which is required for binding to the cell membrane.¹ GPC1 has two N-glycans bound to the N-terminal region and three heparan sulfate chains bound to the C-terminal region, which may actively contribute to its function in the cellular context.¹

GPC1 is mainly expressed during embryonic development and is involved in the development of the nervous and skeletal systems. However, it is absent or expressed at very low levels in adult tissues.¹⁻¹⁰ A distinctive feature of GPC1 is its high expression in pancreatic adenocarcinoma (PDAC), glioblastoma (GBM), prostate cancer (PC) and esophageal squamous-cell carcinomas (ESCC).¹⁻¹⁰ On the other hand, it is not expressed in benign neoplastic lesions or healthy adult tissues.¹⁻¹⁰ In the context of the above mentioned tumors, GPC1 is actively involved in tumorigenesis and is significantly associated with poor prognosis.¹⁻¹⁰ Its localization on the cell membrane and its expression mainly restricted to malignant cells, make GPC1 a “tumor-associated antigen” (TAA) of interest as a target for targeted therapies and for early diagnosis through antibody-based approaches.¹⁻¹⁰

The treatment of hematologic and solid neoplasms with antibodies whose cytotoxic activity targets neoplastic cells has become increasingly important with the development, validation, and introduction into clinical practice of a large number of antibodies for targeted therapies.¹¹⁻¹⁴ The large number and diversity of potential antibody-based targeted approaches reflects the unique versatility of antibody-based platforms for cancer therapy development.¹¹⁻¹⁴

The antitumor effects triggered by therapeutic antibodies are the result of various mechanisms, which depend on the type of the target antigen, the target cell, and the nature of the interactions between the antigen-binding fragment and the crystallizable antibody fragment (Fc) with the target antigen and effector cells, respectively.¹⁴ One of the possible mechanisms of action is the functional neutralization of the target antigen. In detail, therapeutic antibodies are capable to target tumor cells or non-tumor cells if localized in the tumor microenvironment by recognizing tumor associated antigens (TAA), growth factor receptors or their ligands, angiogenic receptors present in the tumor vasculature or their ligands, and molecules acting on immune checkpoints.¹⁴ Antitumor activity depends on the level of expression and turnover of the target antigen expressed by tumor cells. These antibodies induce conformational changes, cause steric disorder, or facilitate internalization and downregulation of surface receptors and inhibit downstream signaling cascades.¹⁴ Several monoclonal antibodies that recognize growth factors and molecules acting on immune checkpoints have already been approved for the treatment of various cancers.¹⁴⁻¹⁹

GPC1 is able to interact with several growth factors such as fibroblast growth factor 2, vascular endothelial growth factor, heparin-binding endothelial growth factor-like growth factor, and transforming growth factor beta, which are known to contribute to tumor cell proliferation, metastasis, and angiogenesis.¹⁻¹⁰ Functional neutralization of GPC1 using a specific monoclonal antibody would provide benefits both in terms of reducing tumor growth, metastasis, and angiogenesis and in remodeling the tumor microenvironment, making it more susceptible to treatments and reducing the state of immunosuppression.

Antibodies can interact with immune system effector cells via Fc and elicit a variety of immune responses, such as: antibody-dependent cell cytotoxicity (ADCC) via the interaction between Fc of the antibody and FC receptors (CD32 and CD89) expressed by neutrophils, and/or the interaction between Fc and CD16 expressed by natural killer cells; antibody-dependent cell phagocytosis (ADCP) by macrophages, which are defined as the main effectors of antibody-mediated therapy because they possess all three classes of FC receptors; complement-dependent cytotoxicity (CDC) via the complement system. Activation of the complement system begins with the interaction between Fc and the C1q protein. This interaction activates the complement cascade, culminating in the formation of the “membrane attachment complex (MAC)” on the surface of the tumor cell, leading to cell lysis. Often, activation of the system may lead to the elimination of tumor cells by ADCC and ADCP, triggering a fruitful cooperation between the different effector pathways.¹⁴

Several anti-GPC1 antibodies are known from the prior art.

For example, EP3617231A1 discloses a number of monoclonal antibodies to human Glypican-1 (GPC1) produced by immunization with a recombinant human GPC1 protein.

WO2021251459A1 discloses humanized antibodies that specifically bind to Glypican-1 (GPC-1).

The antigen used in WO2021251459A1 to produce the humanized antibodies is a purified Glypican-1, a Glypican-1 expressing cell, or a Glypican-1 containing lipid membrane.

WO2016112423A1 discloses a series of small epitopes within the GPC-1 protein that are preferably targeted by binding entities, including antibodies. The identification of such GPC-1 epitopes stems from the need to provide convenient, reliable and accurate tests for diagnosing prostate cancer, especially during the early stages of the disease.

### SUMMARY OF THE INVENTION

The present invention now provides a novel anti-GPC1 monoclonal antibody as defined in appended claim 1, which exhibits a number of unique properties making it particularly suitable and advantageous for use in cancer therapy and diagnosis.

Advantageous embodiments of the invention are defined in the dependent claims.

The anti-GPC1 monoclonal antibody of the invention was obtained by the hybridoma technique, by immunization of mice with a recombinant antigen consisting of the last 70 amino acids of the —COOH terminal region of human GPC1. In detail, antigen production was locally induced in muscle cells of mice by in vivo electroporation of a vector comprising the DNA sequence encoding for the last 70 amino acids of the —COOH terminal region of human GPC1.

The amino acid sequence of human GPC1 is available from the UniProt database under accession number P35052. The amino acid sequence of the GPC1 antigen used to produce the anti-GPC1 monoclonal of the invention is as follows:

(SEQ ID NO: 1)
GSGDGCLDDLCSRKVSRKSSSSRTPLTHALPGLSEQEGQKTSAASCPQPP
TFLLPLLLFLALTVARPRWR

To the inventors' knowledge, the prior art does not disclose the use of the aforementioned 70-amino acids GPC1 antigen to produce an anti-GPC1 monoclonal antibody.

As will be shown in detail in the experimental examples, the monoclonal antibody of the invention exhibits the following unique biological properties:
ability to specifically recognize the GPC1 protein in vitro and ex vivo;
ability to specifically recognize the GPC1 protein in vivo;
ability to induce complement dependent cytotoxicity in vivo;
-ability to reduce GPC1-expressing tumor masses in vivo;
-ability to specifically target polymeric nanoparticles in vivo; and
ability to increase the efficacy of polymeric nanoparticles treatment in vivo through specific active targeting.

In the present description, the anti-GPC1 monoclonal antibody of the invention is also designated as “anti-GPC1 C”.

The term “monoclonal antibody” includes both a full-length immunoglobulin and an antigen-binding fragment of a full-length immunoglobulin, such as a F(ab′)2 or Fab, which retains the same structure of the antigen-binding site and, as a consequence, the same binding abilities as the full length immunoglobulin. Another aspect of the invention is a nucleic acid construct encoding the aforementioned monoclonal antibody.

Yet another aspect of the invention is a host cell transformed with the aforementioned nucleic acid construct.

A further aspect of the invention is a pharmaceutical preparation comprising the aforementioned monoclonal antibody in combination with a pharmaceutically acceptable excipient, diluent and/or vehicle.

Still another aspect of the invention is a method of recombinantly producing the aforementioned monoclonal antibody.

Further aspects of the invention are the uses of the aforementioned monoclonal antibody in tumor diagnosis and therapy.

### BRIEF DESCRIPTION OF THE DRAWINGS

The invention is now described in more detail, by referring to the appended drawings, in which:

FIG. 1A,B shows the results of the flow cytometry analysis of GPC1 expression in different cell lines using anti-GPC1 C or a commercial polyclonal anti-GPC1 antibody (FIG. 1A: BXPC3, JURKAT; FIG. 1B: T98G, U87MG).

FIG. 2A,B shows representative immunofluorescence images of the GPC1 protein in BXPC3, JURKAT (FIG. 2A, scale bar corresponds to 25 μm, DAPI (4′,6-diamidino-2-phenylindole) was employed for nuclear staining), U87MG, T98G (FIG. 2B, scale bar corresponds to 50 μm) cell lines using anti-GPC1 C and a commercial polyclonal anti-GPC1 antibody as reference.

FIG. 3 shows representative immunofluorescence images of the GPC1 protein in glioblastoma (GBM) tissue, liver tissue, and lung tissue, using anti-GPC1 C and a commercial polyclonal anti-GPC1 antibody as reference. Scale bar corresponds to 50 μm.

FIG. 4 shows representative immunofluorescence images of the GPC1 protein in the subcutaneous murine model of pancreatic adenocarcinoma (PDAC) using anti-GPC1 C and a commercial polyclonal anti-GPC1 antibody as reference. Scale bar corresponds to 25 μm.

FIG. 5A,B,C,D,E,F,G shows representative images of in vivo anti-GPC1 C biodistribution studies in a mouse model subcutaneously injected with BXPC3 pancreatic cancer cells and injected i.v. with anti-GPC1 C conjugated with Cy5.5.

FIG. 5A shows representative images of whole-body scan in prone and supine position of a mouse in the context of in vivo anti-GPC1 C biodistribution studies in a mouse model obtained by subcutaneously injecting BXPC3 pancreatic cancer cells and subsequently injected i.v. with anti-GPC1 C conjugated with Cy5.5. Whole-body scan of a representative mouse in prone and supine position is shown. Fluorescence intensity images were acquired at the indicated time post-injection and are displayed in efficiency. Green circles enclose the tumor mass.

FIG. 5B shows whole mouse body scan representative images of in vivo IgM pool biodistribution studies in a mouse model obtained by subcutaneously injecting BXPC3 pancreatic cancer cells and subsequently injected i.v. with murine IgM pool conjugated with Cy5.5 as control. Whole-body scan of a representative mouse in prone and supine position is shown. Fluorescence intensity images were acquired at the indicated time post-injection and are displayed in efficiency. Green circles enclose the tumor mass.

FIG. 5C shows whole mouse body scan representative images of phosphate buffered saline (PBS) injection in negative control experiment for in vivo biodistribution studies in a mouse model obtained by subcutaneously injecting BXPC3 pancreatic cancer cells. Whole-body scan of a representative mouse in prone and supine position is shown. Fluorescence intensity images were acquired at the indicated time post-injection and are displayed in efficiency. Green circles enclose the tumor mass.

FIG. 5D shows histograms reporting fluorescence intensity values of in vivo biodistribution studies of anti-GPC1 C (1 nmol Cy5.5, 38 μg anti-GPC1 C), compared with the IgM pool and PBS, injected i.v. in a mouse model obtained by subcutaneously injecting BXPC3 cells. Fluorescence intensity images were acquired at the indicated time post-injection and are displayed in efficiency. Data are reported as average efficiency mean±SD (n=4). P-value was calculated using the t-test, for the t-test, anti-GPC1 C was used as reference for each time point. ns: ≥0.05; *: 0.05<p≤0.01; **: 0.01<p≤0.001.

FIG. 5E shows ex vivo images taken 96 h after the administration of anti-GPC1 C, IgM pool and PBS injected i.v. in a mouse model obtained by subcutaneously injecting BXPC3 cells.

FIG. 5F shows histograms reporting fluorescence intensity values of ex vivo images taken 96 h after the administration of anti-GPC1 C. IgM pool and PBS injected i.v. in a mouse model obtained by subcutaneously injecting BXPC3 cells. Fluorescence intensity images were acquired at 96 h and are displayed in efficiency. Data are reported as mean±SD (n=4). P-value was calculated using the t-test. ns: ≥0.05.

FIG. 5G shows representative ex vivo immunofluorescence images of IgM deposition (after injection of anti-GPC1 C or IgM pool or PBS) in the mouse model obtained by subcutaneously injecting BXPC3 cells. Blue represents nuclei and green represents IgMs. Scale bar corresponds to 25 μm.

FIG. 6 shows images of the histological evaluation of tumor tissue by hematoxylin eosin staining (after injection of anti-GPC1 C or PBS) in the mouse model obtained by subcutaneously injecting BXPC3 cells. Tumor sections were analyzed 96 h after the administration of anti-GPC1 C. The nuclei are stained in purple, the cytoplasm is stained in pink. Scale bar correspond to 100 μm.

FIG. 7A,B shows representative ex vivo immunofluorescence images of the presence of NK cells (CD14-expressing cells, FIG. 7A) and macrophages (CD56-expressing cells, FIG. 7B) deposition in the tumor masses of the mouse model obtained by subcutaneously injecting BXPC3 cells (after injection of anti-GPC1 C or IgM pool or PBS). Blue represents nuclei and green represents the signal related to the immunological cell biomarkers (CD14 and CD56, respectively). Scale bar corresponds to 100 μm.

FIG. 8A,B,C shows representative ex vivo immunofluorescence images of complement system (C1q: FIG. 8A; C3: FIG. 8B; C9: FIG. 8C) deposition in tumor masses of the mouse model obtained by subcutaneously injecting BXPC3 cells. Blue represents nuclei and green represents the signal related to the complement system effectors (C1q, C3, C9, respectively). Scale bar corresponds to 100 μm.

FIG. 9 is a graph showing the anti-GPC1 C therapeutic effect in a mouse model obtained by subcutaneously injecting BXPC3 cells. Tumor-bearing mice received i.p 76 μg of anti-GPC1 C (light blue line) until day 42. Experimental endpoint was set up at day 50. Untreated mice (orange line) were used as controls. Data were visualized using Kaplan-Meier analysis, p-value of 0.00016 was calculated using the log rank test.

FIG. 10 is a line graph that recapitulates the tumor growth trend of the experiment reported in the previous FIG. 9. In orange the untreated group (PBS), in blue the group treated with anti-GPC1 C. Data are represented as mean±standard error (SE).

FIG. 11 shows the results of in vivo biodistribution studies of chitosan nanobubbles (CS-NBs) conjugated or not conjugated with the anti-GPC1 C in a mouse model obtained by subcutaneously injecting BXPC3 cells.

FIG. 11A shows a representative biodistribution of chitosan nanobubbles (CS-NBs) conjugated with the anti-GPC1 C antibody (CS NBs-C), and conjugated with Cy5.5 to allow in-vivo detection (Cy5.5-CS NBs-C) (1 nmol Cy5.5, 10E11 NBs), injected i.v. in a mouse model obtained by subcutaneously injecting BXPC3 cells. Whole-body scan of a representative mouse in prone and supine position is shown. Fluorescence intensity images were acquired at the indicated time post-injection and are displayed in efficiency. Green circles enclose the tumor mass.

FIG. 11B shows a representative biodistribution of chitosan nanobubbles not conjugated with the anti-GPC1 C antibody (CS NBs) and conjugated with Cy5.5 to allow in-vivo detection (Cy5.5-CS NBs) (1 nmol Cy5.5, 10E11 NBs), injected i.v. in a mouse model obtained by subcutaneously injecting BXPC3 cells. Whole-body scan of a representative mouse in prone and supine position is shown. Fluorescence intensity images were acquired at the indicated time post-injection and are displayed in efficiency. Green circles enclose the tumor mass.

FIG. 11C shows a representative example of phosphate buffered saline (PBS) i.v. injection in a negative control experiment for in vivo biodistribution studies in a mouse model obtained by subcutaneously injecting BXPC3 cells. Whole-body scan of a representative mouse in prone and supine position is shown. Fluorescence intensity images were acquired at the indicated time post-injection and are displayed in efficiency. Green circles enclose the tumor mass.

FIG. 11D shows histograms reporting fluorescence intensity values of in vivo tumor accumulation of chitosan nanobubbles conjugated or not conjugated with the anti-GPC1 antibody C when injected i.v. in a mouse model obtained by subcutaneously injecting BXPC3 cells. In particular, it is shown the biodistribution of Cy5.5-CS NBs-C (1 nmol Cy5.5), compared with Cy5.5-CS NBs (1 nmol Cy5.5) and PBS. Fluorescence intensity images were acquired at the indicated time post-injection and are displayed in efficiency. Data are reported as average efficiency mean±SD (n=4). P value was calculated using the t-test, Cy5.5-CS NBs-C was used as reference for each time point.

FIG. 11E shows representative images from the ex vivo biodistribution analysis of Cy5.5-CS NBs-C vs Cy5.5-CS NBs and PBS. Ex vivo imaging was performed 96 h after administration of Cy5.5-CS NBs-C. Cy5.5-CS NBs, and PBS.

FIG. 11F histograms reporting ex-vivo organ accumulation of Cy5.5-CS NBs-C compared to Cy5.5-CS NBs and PBS when injected in a mouse model obtained by subcutaneously injecting BXPC3. In particular, it is reported the ex-vivo biodistribution of Cy5.5-CS NBs-C (1 nmol Cy5.5, 1011 NBs), compared with Cy5.5-CS NBs (1 nmol Cy5.5, 1011 NBs) and PBS (as negative control). Fluorescence intensity images were acquired at 96 h and are displayed in efficiency. Data are reported as mean±SD (n=4). P value was calculated using the t-test. ns: ≥0.05.

FIG. 11G shows representative images from the immunofluorescence (IF) evaluation of the localization of Cy5.5-CS NBs-C and Cy5.5-CS NBs within the tumor. In red the signal of Cy5.5 related to the NBs and in blue the nuclei. NBs are indicated with the white arrows. Scale bar 25 μM.

FIG. 12 is a line graph that recapitulates the tumor growth trend. In green the untreated group, in light blue chitosan nanobubbles loaded with doxorubicin (doxo-CS NBs), and in blue doxo-CS NBs-C. The amount of doxorubicin loaded nanobubbles used for the treatments in each group corresponded to 2 mg/kg per week of doxorubicin for each different treatment modality. Data are represented as mean±SD.

FIG. 13 shows the Kaplan-Meier curve representing the in vivo therapeutic effect of doxorubicin (doxo) at the dose of 2 mg/kg per week, doxo-CS NBs at the dose corresponding to 2 mg/kg of doxorubicin per week and doxo-CS NBs-C at the dose corresponding to 2 mg/kg of doxorubicin per week in a mouse model obtained by subcutaneously injecting BXPC3 cells. Kaplan-Meier curves of untreated group, doxo 2 mg/kg per week, doxo-CS NBs at the dose corresponding to 2 mg/kg of doxorubicin per week and doxo-CS NBs-C at the dose corresponding to 2 mg/kg of doxorubicin per week are statistically different. P-value was calculated using the log-rank test. Colored areas correspond to 95% confidence interval.

FIG. 14 shows the Kaplan-Meier curves of: doxo-CS NBs in comparison with doxo 2 mg/kg (upper-left panel); doxo-CS NBs-C in comparison with doxo 2 mg/kg (upper-right panel); doxo-CS NBs in comparison with doxo-CS NBs-C (lower panel). The survival data from the four treatments were evaluated by Kaplan-Meier curves to compare each treatment with one at time of the others. All the comparisons showed a statistical difference. P-value was calculated using the log-rank test. Colored areas correspond to 95% confidence interval.

FIG. 15 shows histograms reporting the fluorescence intensity values of the in vivo tumor accumulation of chitosan nanobubbles (CS NBs) when injected i.v. in a mouse model obtained by subcutaneously injecting U87MG cells. PBS i.v. injection was performed as negative control experiment for the in vivo biodistribution studies (PBS is reported as control in the Figure). In particular, it is shown the biodistribution of Cy5.5-CS NBs-C (1 nmol Cy5.5), compared with Cy5.5-CS NBs (1 nmol Cy5.5) and PBS. Fluorescence intensity images were acquired at the indicated time post-injection and are displayed in efficiency. Data are reported as average efficiency mean±SD (n=4). P value was calculated using the t-test, Cy5.5-CS NBs-C was used as reference for each time point.

FIG. 16 shows ex-vivo fluorescence microscopy images of three explanted tumour masses and three liver cryosections (mice n.1, n.2, n.3) obtained from Cy5.5-CS NBs-C injections into mice. Nuclei are reported in blue and Cy 5.5 in red to visualize NBs-B-Cy 5.5. Scale bar corresponds to 50 μm.

### DETAILED DESCRIPTION OF THE INVENTION

The monoclonal antibody of the invention is a monoclonal antibody specifically directed against the tumor-associated antigen Glypican-1 (GPC1).

The specificity was confirmed by ELISA and cytofluorometric analysis showing the absence of unspecific interactions, in particular on cells like T-cell line Jurkat, not expressing GPC1. Moreover, ELISA demonstrated the specificity for GPC1 with respect to other similar molecule, like GPC3.

This monoclonal antibody is of the IgM isotype. As is well known, a full-length IgM molecule is composed, like all immunoglobulins, of two heavy chains and two light chains. IgM light chains are of the k or the λ type. IgM heavy chains are of the μ type and contain a constant Cμ region consisting of four Ig domains. IgMs exists in two possible conformations, namely the monomeric, membrane form and the pentameric, secreted form. In the pentameric form, IgMs are organised into a complex of five monomers held together by disulphide bridges, which are formed in the terminal sequences of the u heavy chains. An additional ˜15 kDa protein is usually attached to the pentamer, i.e. the so-called J chain, which is linked to the terminal sequence by disulphide bridges and stabilizes the entire complex.²⁰ The scope of the invention includes both the monomeric and the pentameric form of the anti-GPC1 IgM monoclonal antibody of the invention. In both embodiments, the monoclonal antibody comprises three heavy chain complementarity determining regions (HCDR1, HCDR2 and HCDR3) and three light chain complementarity determining regions (LCDR1, LCDR2 and LCDR3), wherein:
HCDR1 includes the amino acid sequence of SEQ ID NO: 3,
HCDR2 includes the amino acid sequence of SEQ ID NO: 5,
HCDR3 includes the amino acid sequence of SEQ ID NO: 7,
LCDR1 includes the amino acid sequence of SEQ ID NO: 9,
LCDR2 includes the amino acid sequence of KVS (wherein K stands for Lysine, V stands for Valine and S stands for Serine), and
LCDR3 includes the amino acid sequence of SEQ ID NO: 12.

In a preferred embodiment, the monoclonal antibody comprises a heavy chain variable region including the amino acid sequence of SEQ ID NO: 13 and a light chain variable region including the amino acid sequence of SEQ ID NO: 14.

Another preferred embodiment is an antigen-binding fragment of the monoclonal antibody of the invention, which retains the ability to bind to glypican-1 (GPC1) and shares the same biological properties as the full-length monoclonal antibody.

In a particularly preferred embodiment, the antigen-binding fragment is selected from the group consisting of F(ab′)2 and Fab.

Another preferred embodiment is the monoclonal antibody of the invention conjugated with a drug, such as for example a cytotoxin or an immunotoxin, so as to form an antibody-drug conjugate (ADC).

Other antibody-drug conjugates (ADC) are known in the art and this type of approach has already led to some ADCs entering the clinic, such as Adcetris (brentuximab vedotin) for the treatment of Hodgkin's Lymphoma and Kadcyla (ado-trastuzumab emtansine) for the treatment of HER2-positive breast cancer.^{21,22}

In the context of the present invention, the drug is conjugated to the antibody moiety through a cleavable or non cleavable linker. Non limiting examples of linkers conventionally used to form ADCs are disulfides, hydrazones or peptides (cleavable), or thioethers (non cleavable). As the drug, any of the antitumor drugs known to the skilled in the art may be selected, for example the cytotoxic molecules doxorubicin and methotrexate.

One of the known main problems with cytotoxic molecules is their hydrophobic nature, which may lead to instability (aggregation) and may affect the in vivo activity of ADC.²³ In order to solve this problem, a polymer such as polyethylene glycol (PEG) can be incorporated into the linker to mask the hydrophobicity of the cytotoxic molecule. Furthermore, by acting on the configuration of the PEG molecule, it is possible to make the ADC more susceptible to the uptake of a high amount of cytotoxic molecules.²³

These strategies for preparing high performing ADCs and selecting the appropriate drug are well known to those skilled in the art, and their implementation is well within their abilities.

In the present invention, a particularly preferred approach is conjugation with emtansine, an inhibitor of tubulin polymerization. In this way, the cytotoxic effect of emtansine on healthy tissues, which is one of the main problems with conventional chemotherapies, shall be focused on the tumor, thanks to the possibility of specifically directing emtansine to the GPC1-expressing cancer cells.

A further preferred embodiment is the monoclonal antibody of the invention conjugated with a drug-loaded polymeric nanoparticle. Nanoparticle-based drug delivery platforms have emerged as suitable vehicles for overcoming pharmacokinetic limitations associated with conventional drug formulations. Nanoparticles proved advantageous at solubilizing therapeutic cargos substantially prolonging the circulation lifetimes of drugs. In the field of oncology, several therapies based on nanoparticles have been developed with properties tuned to improve drug delivery to the tumor, overcoming the impairments caused by chemotherapy and radiotherapy.^{2,24} In addition, several nanoparticles have already been introduced into clinical practice, such as liposomal doxorubicin (Doxil) and nanoparticle albumin-bound paclitaxel (Abraxane).^{25,26} Polymeric nanoparticles offer several advantages: they are biodegradable, they allow controlled release of their cargo in a specific part of the body, they protect the drugs they contain from degradation and increase their solubility, they can be modified on the surface with specific molecules, they can be loaded with large amounts of drugs, they protect healthy tissues from the toxic effects of the anticancer drugs they contain, they offer excellent in vitro and in vivo stability, and they have an extended blood circulation time.^{27,28} One of the most commonly used polymers for nanotechnology approaches to drug delivery is chitosan.²⁹⁻³¹ Chitosan is a hydrophilic, positively charged polysaccharide formed by alkaline hydrolysis of chitin, a natural component of the cell walls of fungi and in the structures of some invertebrate animals and fish. Chitosan is non-toxic, biocompatible, its biodegradation produces non-toxic oligosaccharides, and it has already been approved by the FDA for tissue engineering and drug delivery.²⁹⁻³¹ It consists of β-(1,4)-linked D-glucosamine (deacetylated) and N-acetyl-D-glucosamine (acetylated) units distributed randomly, the number and type of which determine its chemical and biological properties. Chitosan is a good choice for nanoparticle delivery because it contains hydroxyl (—OH) and amine (—NH₂) functional groups for the addition of crosslinking agents and has a positive charge that allows binding to cells, which increases cellular uptake and thus enables drug release within cells.²⁹⁻³¹ In the last years, chitosan-based nanoparticles have been used in vitro and in vivo to investigate their effect on several cancers.²⁹⁻³¹ This nanoformulation showed increased uptake from cancer cells and, once loaded with drugs, as well as improved cytotoxic activity, increased half-life of drug circulation, and higher inhibition of tumor growth compared to free drugs.²⁹⁻³¹ Other polymers commonly used to prepare polymeric nanoparticles are Poly(glycolic acid) (PGA), poly(lactic acid) (PLA), poly(lactic-co-glycolic acid) (PLGA). PGA, PLA and PLGA are polymers from the polyester family. They were developed in the 1960s for surgical implants and tissue repair, but soon after they were used for controlled release of drugs.³⁰ They are biocompatible, non-toxic, and biodegradable due to hydrolytic cleavage at the ester bond between lactic acid and/or glycolic acid. In general, polymeric nanoparticles with a size between 200 and 300 nm can be easily prepared, an ideal size for a drug delivery approach, as they optimize internalization by tumor cells and increase retention time in the blood thanks to the effect of increased permeability and retention (EPR effect).³² Specifically concerning the conjugation of the anti-GPC1 monoclonal antibody of the invention with polymeric nanoparticles, it should be emphasized that conventional chemotherapies are still used in clinical practice for the treatment of solid tumors, despite the need to develop new and more effective therapies.^{2,24} Very often, the lack of efficacy of these chemotherapeutic agents is due to the difficulty in reaching the tumor or to the presence of resistance mechanisms in the tumor microenvironment. Another important problem is the possible occurrence of “off-side toxicity” phenomena.^{2,24} Therefore, conjugation of drug-loaded polymeric nanoparticles with the anti-GPC1 monoclonal antibody of the invention may enhance the ability of the drug-loaded nanoparticles to reach the tumor site. Any drug conventionally used in antitumor therapy can be used for this purpose. Non-limiting examples include paclitaxel and anthracyclines such as doxorubicin for the treatment of PDAC, and temozolamide for the treatment of GBM.^{33,34} Any other chemotherapeutic agents conventionally used for cancer treatment can also be loaded into polymeric nanoparticles to treat other specifically GPC1-expressing tumors, such as prostate cancer and squamous cell carcinoma of the esophagus.

Strategies for preparing drug-loaded polymeric nanoparticles, as well as their conjugation with antibody moieties and the selection of the appropriate drug are well known to those skilled in the art, and their implementation is well within their abilities.

Another aspect of the invention is the monoclonal antibody of the invention for use in the therapeutic treatment of a GPC1-expressing tumor. The GPC1-expressing tumor is preferably selected from the group consisting of pancreatic adenocarcinoma, glioblastoma, prostate cancer, and squamous cell carcinoma of the esophagus.

For therapeutic applications, the monoclonal antibody of the invention is provided in a pharmaceutical preparation.

A pharmaceutical preparation comprises the monoclonal antibody of the invention as the active ingredient, and at least one pharmaceutically acceptable excipient, vehicle or diluent. In a preferred embodiment, the pharmaceutical preparation is designed to be administered intravenously, intramuscularly, subcutaneously, intraperitoneally, intranasally, parenterally or as an aerosol.

Examples of suitable pharmaceutical carriers, excipients and/or diluents are well known in the art and include phosphate buffered saline solutions, water, emulsions, such as oil/water emulsions, various types of wetting agents, sterile solutions etc. Pharmaceutical preparations comprising such carriers can be formulated by conventional methods well known in the art. Pharmaceutical preparations are administered to the subject at a suitable dose, i.e. a dose that contains an amount of the active ingredient which is sufficient to substantially inhibit tumor growth or metastasis of the tumor. As is well known in the medical arts, the suitable dose depends upon many factors, including patient's size, body surface area, age, the compound to be administered, sex, time and route of administration, general health, and whether other drugs are to be concurrently administered. The selection of the suitable dose for each patient is well within the skills of the skilled practitioner.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

To produce the monoclonal antibody of the invention recombinant methods known in the art are employed. The nucleic acid encoding the monoclonal antibody of the invention is introduced into a host cell and expressed using materials and procedures known in the art.

More in particular, a nucleic acid molecule encoding the monoclonal antibody of the invention is inserted into an appropriate expression vector using standard ligation techniques. The vector is typically selected to be functional in the host cell to be employed. A nucleic acid molecule encoding the monoclonal antibody of the invention may be amplified/expressed e.g., in prokaryotic, yeast, insect (baculovirus systems) and/or eukaryotic host cells. Typically, expression vectors used in any host cells will contain one or more of the following components: a promoter, one or more enhancer sequences, an origin of replication, a transcriptional termination sequence, a complete intron sequence containing a donor and acceptor splice site, a leader sequence for secretion, a ribosome binding site, a polyadenylation sequence, a poly-linker region for inserting the nucleic acid encoding the polypeptide to be expressed, and a selectable marker.

The expression vector is inserted into the host cell using standard transformation or transfection techniques. Methods for transformation or transfection, culture, amplification, screening as well as product production and purification are known in the art.^{35,36}

Yet another aspect of the invention is an in vitro method of detecting or diagnosing a GPC1-expressing tumor in a subject. The method comprises contacting a detection reagent comprising the monoclonal antibody of the invention with a sample from the subject (e.g. a cell sample or a tissue sample) and then detecting binding of the detection reagent with the sample. As GPC1 is selectively expressed by tumors and not by benign neoplastic lesions or healthy cells or tissues, binding is indicative of the presence of a tumor. Suitable means for detection are reagents conventionally used in immuno- or nucleic acid-based diagnostic methods. The monoclonal antibody of the invention is, for example, suited for use in immunoassays in which it is employed in a liquid phase or is bound to a solid phase carrier. Examples of immunoassays suitable for use with the monoclonal antibody of the invention are competitive and non-competitive immunoassays in either a direct or indirect format. Examples of such immunoassays are the radioimmunoassay (RIA), the ELISA immunoassay, flow cytometry and the Western blot assay. As is well known to the skilled in the art, for use in such methods the monoclonal antibody of the invention is usually labeled. There are many different labels and methods of labeling known to those of ordinary skill in the art. Non limiting examples include enzymes, radioisotopes, colloidal metals, fluorescent compounds, chemiluminescent compounds, and bioluminescent compounds. When employed in the context of imaging tools such as Gadolinium (III) based probes or nanoparticles,³⁷ the monoclonal antibody of the invention even allows early tumor diagnosis.

The following experimental section is provided by way of illustration only and is not intended to limit the scope of the invention as defined in the appended claims.

### EXPERIMENTAL SECTION

### Example 1

The DNA sequence of the GPC1 antigen used for the immunization of mice encodes for the last 70 amino acids in the —COOH terminal region of the protein. The amino acid sequence of human GPC1 is available from the UniProt database under accession number P35052.

A eukaryotic vector encoding for the last 70 amino acids in the —COOH terminal region of the protein was produced and used for in vivo transfection of muscle cells by electroporation.

The amino acid sequence of the GPC1 antigen used for immunization of mice is as follows:

(SEQ ID NO: 1)
GSGDGCLDDLCSRKVSRKSSSSRTPLTHALPGLSEQEGQKTSAASCPQPP
TFLLPLLLFLALTVARPRWR

Hybridoma cells were then obtained through a standard approach. Several clones were tested for their ability to produce in culture medium an antibody (IgG or IgM) able to specifically target GPC1; these data derived from both ELISA and cytofluorimetric analysis.

The monoclonal antibody of the invention (“anti-GPC1 C”) was produced in a cell supernatant and purified initially by affinity chromatography and then by anionic exchange chromatografy. Anti-GPC1 C is an IgM and it is characterized by the following heavy chain and light chain variable regions, respectively:

IGHV (heavy chain variable region):
(SEQ ID NO: 13)
QVQLQQSDAELVKPGASVKISCKASGYTFTDHAIHWVKQKPEQGLEWIGY
ISPGNGDIKYNEKFKGKATLTADKSSSTAYMQLNSLTSEDSAVYFCKRYA
YWGQGTLVTVSA
IGLV (light chain variable region):
(SEQ ID NO: 14)
DVLMTQTPLSLPVSLGDQASISCRSSQSIVHSNGNTYLEWYLQKPGQSPK
LLIYKVSNRFSGVPDRFSGSGSGTDFTLKISRVEAEDLGVYYCFQGSHVP
WTFGGGTKLEIKP

The inventors further analyzed the heavy chain and light chain variable regions to identify their complementarity determining regions and framework regions, whose amino acid sequences are disclosed below:

IGHV

| | | SEQ |
|---|---|---|
| | | ID |
| Region | Amino acid sequence | NO: |
| FR1 | QVQLQQSDAELVKPGASVKISCKAS | 2 |
| CDR1 | GYTFTDHA | 3 |
| FR2 | IHWVKQKPEQGLEWIGY | 4 |
| CDR2 | ISPGNGDI | 5 |
| FR3 | KYNEKFKGKATLTADKSSSTAYMQLNSLTSEDSAVYFC | 6 |
| CDR3 | KRYAYWGQGTLVTVSA | 7 |

IGLV

| | | SEQ |
|---|---|---|
| Re- | | ID |
| gion | Amino acid sequence | NO: |
| FR1 | DVLMTQTPLSLPVSLGDQASISCRSS | 8 |
| CDR1 | QSIVHSNGNTY | 9 |
| FR2 | LEWYLQKPGQSPKLLIY | 10 |
| CDR2 | KVS(Lys-Val-Ser) | n.a.* |
| FR3 | NRFSGVPDRFSGSGSGTDFTLKISRV | 11 |
| | EAEDLGVYYC | |
| CDR3 | FQGSHVPWTFGGGTKLEIKP | 12 |

*n.a. = not applicable

### Example 2

### Anti-GPC1 C Recognizes the GPC1 Protein In Vitro and Ex Vivo

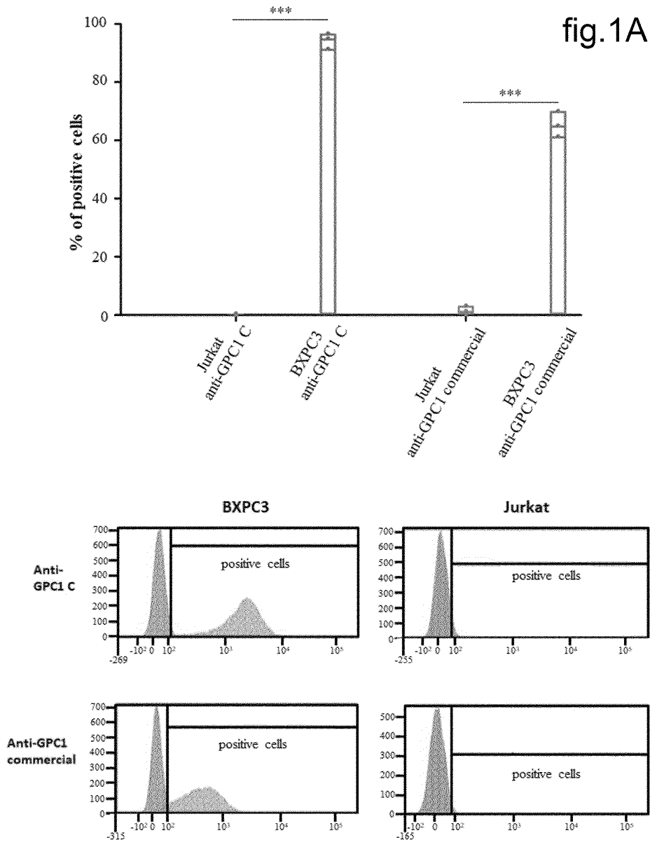

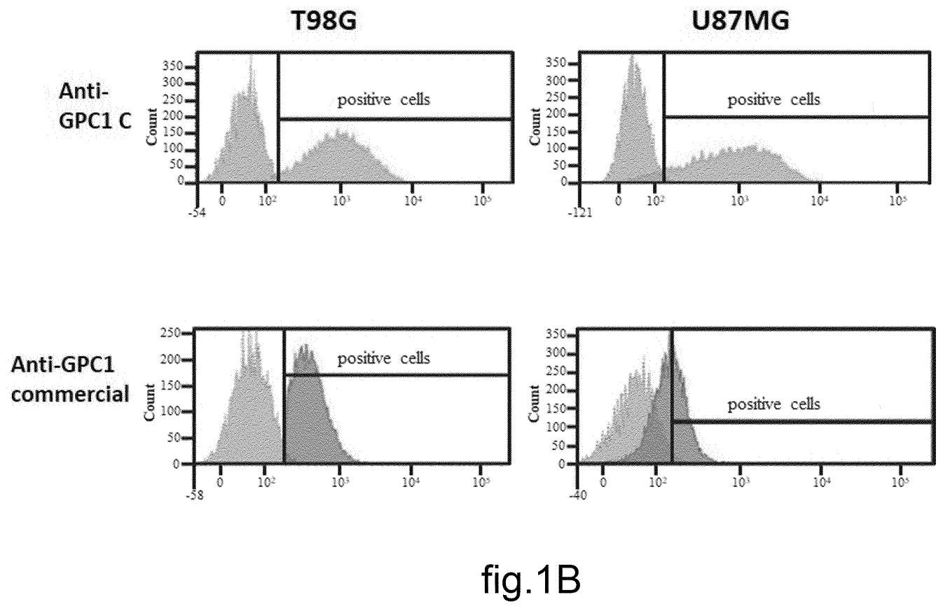

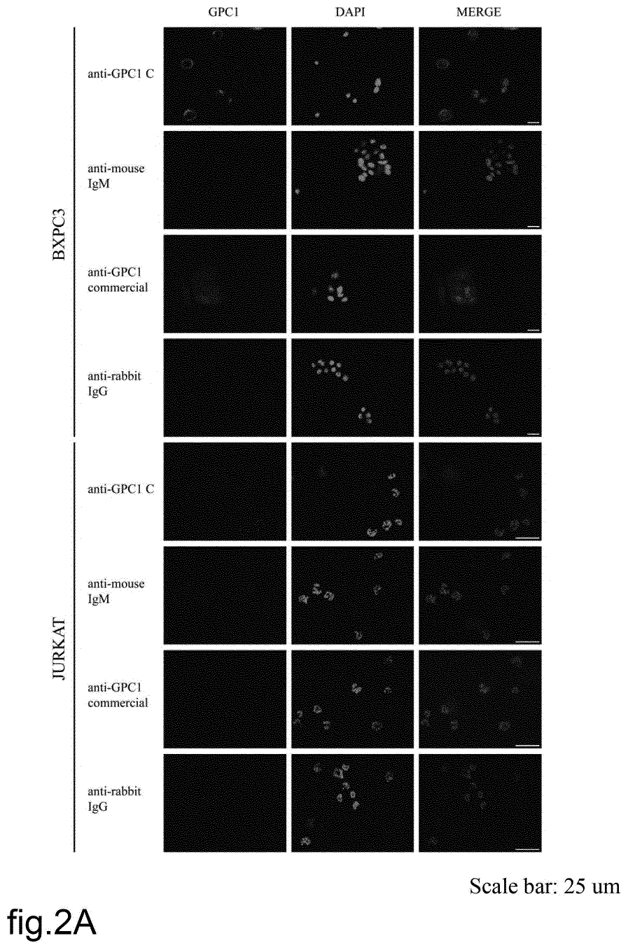

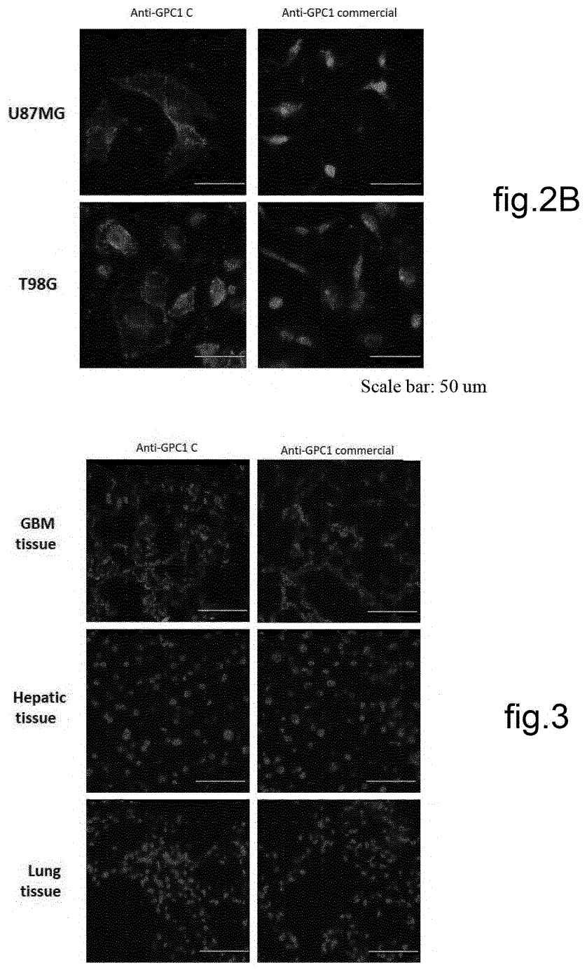

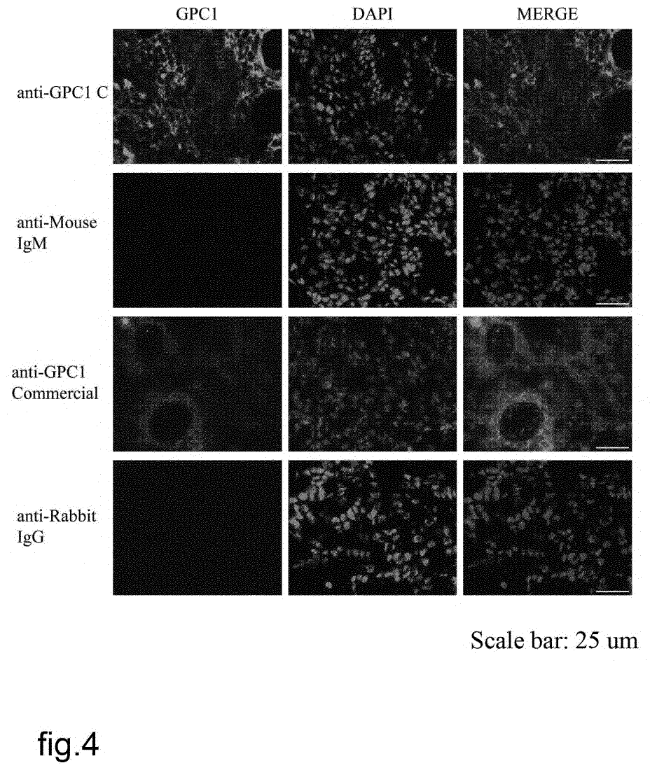

a) Anti-GPC1 C was shown to specifically recognize the GPC1 protein by flow cytometry in the GPC1-expressing tumor cell line model cells BXPC3 cell line cells (PDAC model tumor model) and U87MG and T98G cell line cells (GBM tumor models). On the other hand, no signals corresponding to the presence of the GPC1 protein was obtained by performing flow cytometry experiments on the JURKAT cell line cells (T ALL model) that are characterized by the complete absence of the GPC1 protein. Of note, the presence or the GPC1 protein expression was defined by using a commercial anti-GPC1 polyclonal antibody that can be employed for evaluation of GPC1 protein expression in the same cell line models (e.g., GPC1 expression in BXPC3, U87MG, T98G cell line models, no expression of the GPC1 protein in the JURKAT cell line model) (FIG. 1A, FIG. 1B).
b) Anti-GPC1 C was also shown to specifically recognize GPC1 protein by immunofluorescence in the GPC1-expressing model cells of tumor cell line BXPC3 and of tumor cell lines U87MG and T98G. Remarkably, the same expression pattern as flow cytometry analysis was obtained by immunofluorescence analysis with anti-GPC1 C. Notably, when immunofluorescence experiments were performed, a signal corresponding to the GPC1 protein was observed in the BXPC3, U87MG, and T98G cell lines, whereas, as expected, no signal was observed when the presence of the GPC1 protein was examined in the JURKAT cells (FIG. 2A, FIG. 2B). These results are consistent with those observed using the commercial antibody used as a control. Thus, the results of immunofluorescence analysis confirmed the ability of anti-GPC1 C to detect the GPC1 protein that was observed when flow cytometry was performed (FIGS. 1A, 1B).
c) Anti-GPC1 C was also shown to specifically recognize GPC1 protein in GBM tissue samples from surgical resection. Indeed, when anti-GPC1 C was used, a specific signal was observed in GBM tissue samples obtained by surgical resection. Moreover, no signal corresponding to GPC1 protein was detected in healthy tissues (liver and lung), where GPC1 protein is known not to be expressed. Remarkably, the same expression pattern was found in analogous immunofluorescence experiments with the commercial anti-GPC1 antibody (FIG. 3).
d) Anti-GPC1 C was also shown to specifically recognize the GPC1 protein in BXPC3 (PDAC model) in tumor masses of xenografts obtained by subcutaneous injection of BXPC3 into athymic nude mice. Remarkably, no signals were obtained when the organs of the mice in which the xenograft model was established were examined. In addition, the GPC1 expression pattern obtained by using the anti-GPC1-C antibody was found to be similar to the pattern obtained when immunofluorescence experiments were performed with the commercial anti-GPC1 antibody (FIG. 4).

### Example 3

### Anti-GPC1 C Recognizes the GPC1 Protein In Vivo

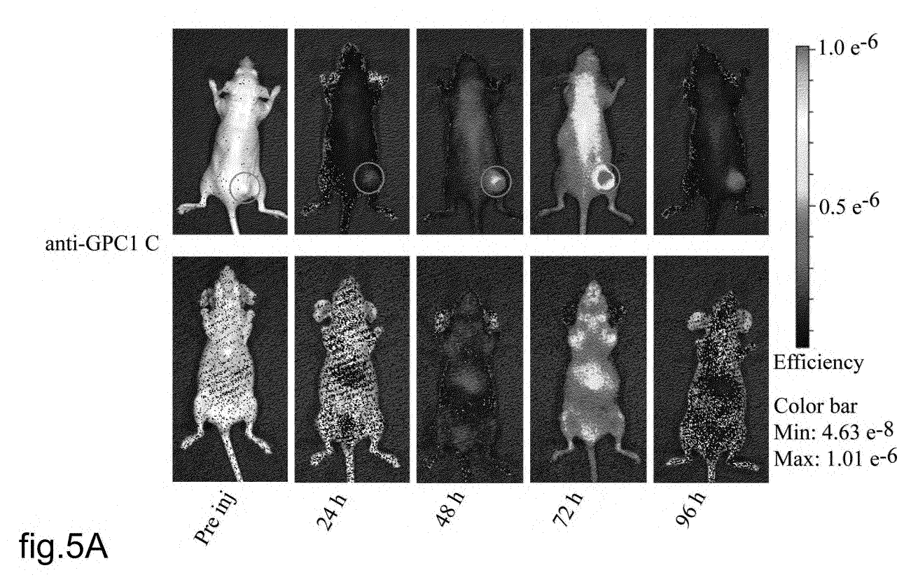

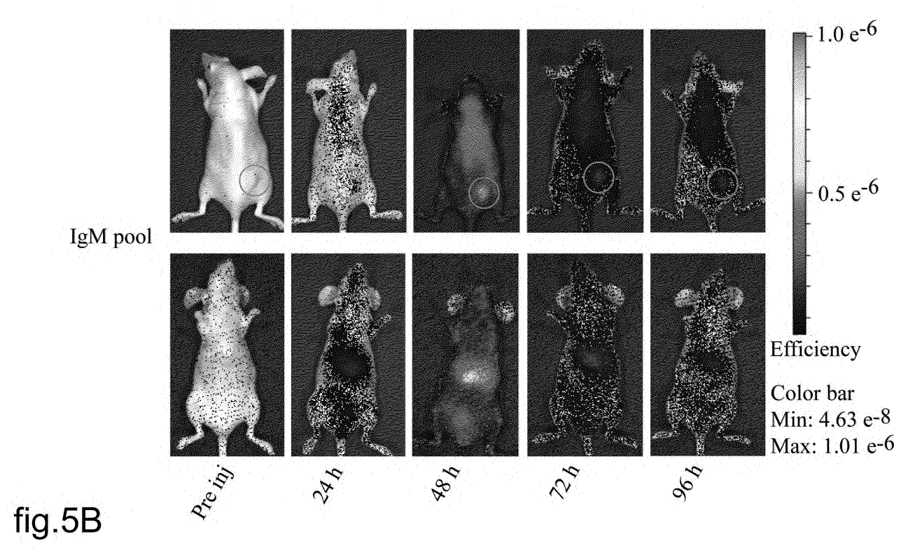

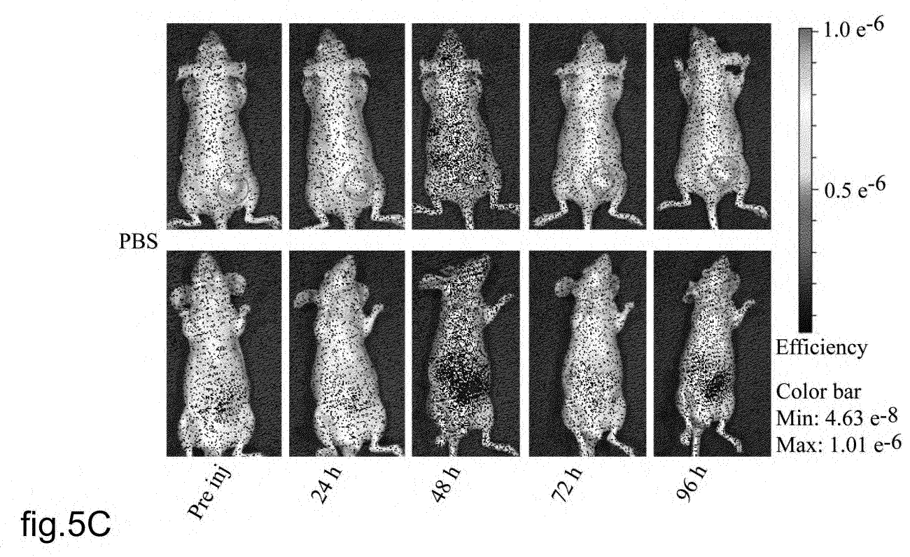

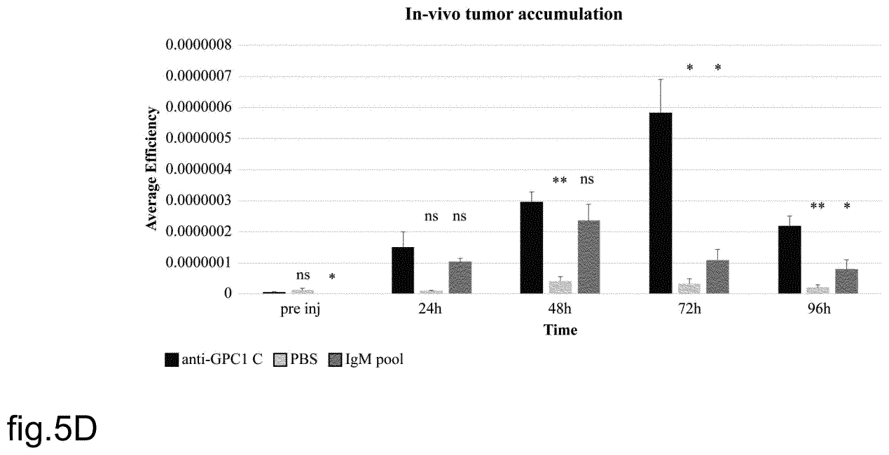

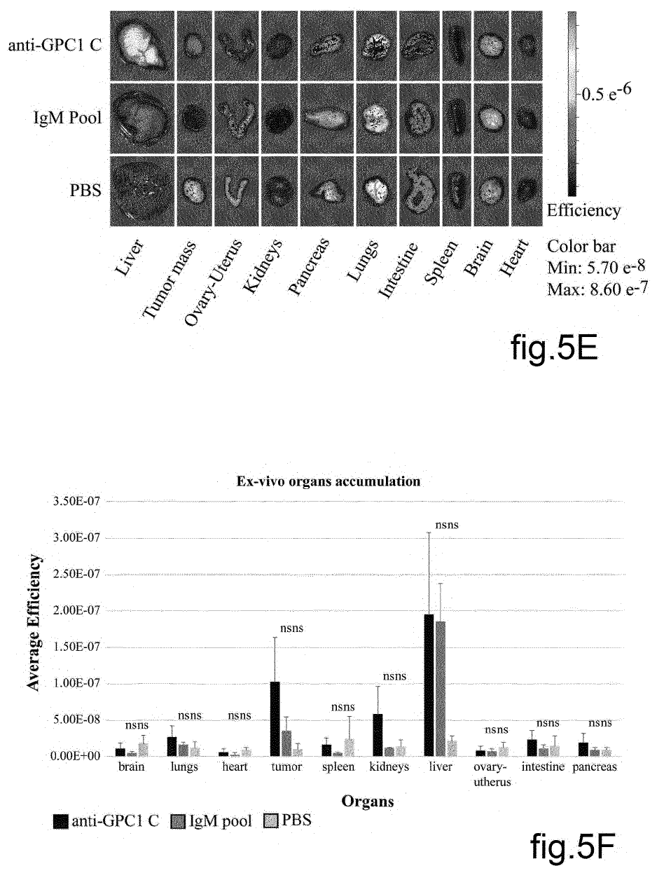

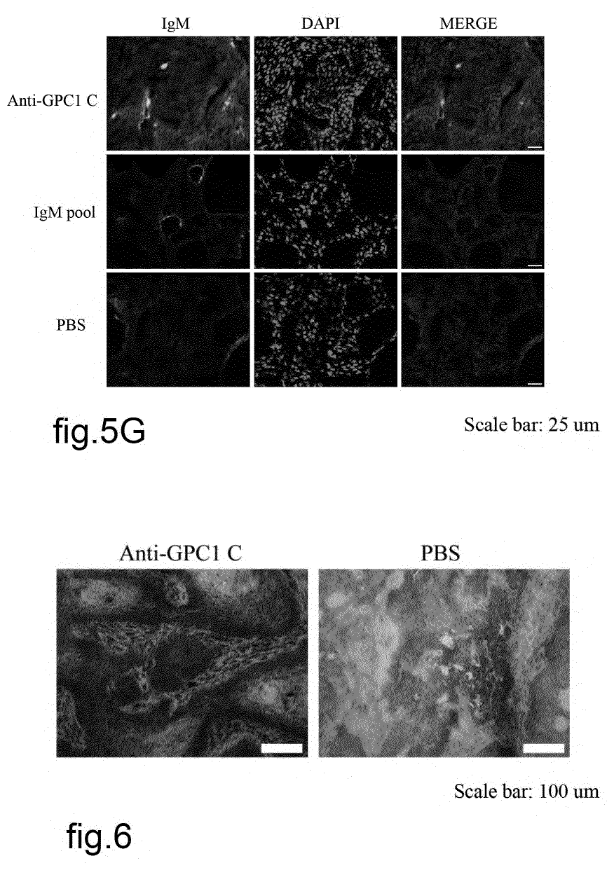

Anti-GPC1 C was shown to target BXPC3 tumor mass in xenograft mouse models obtained by subcutaneous injection of BXPC3 cells into athymic nude mice. Specifically, in vivo time course experiments with signal detection from 0 to 96 hours and signal evaluation every 24 hours were performed to evaluate the ability to target tumor mass and the overall biodistribution of anti-GPC1 C when injected into the mouse tail vein after conjugation with Cy5.5 for detection using the VIVOVISION IVIS®Lumina (Xenogen) in vivo imaging device (FIGS. 5A, B, C, D, E, F, G).

In these experiments, the accumulation of anti-GPC1 C on the BXPC3 tumor mass was assessed from the time point of 24 hours, and the signal was found to be maintained up to 96 hours, with a peak of accumulation at 72 hours. These results indicate that anti-GPC1 C is capable of reaching BXPC3 tumor mass when injected in vivo. A clear signal corresponding to the presence of anti-GPC1 C was also seen in the kidneys and liver, as expected, as these organs are responsible for body excretion (FIGS. 5A and 5D). Remarkably, a different distribution pattern was obtained when a pool of murine IgM was used as a control, with a more rapid peak of accumulation at 48 hours and no detectable signal at 96 hours (FIGS. 5B and 5D).

Moreover, as expected, no signal was obtained when PBS was intra-vein injected as further negative control (FIGS. 5C and 5D).

Ex vivo analysis performed 96 hours after administration using the VIVOVISION IVIS®Lumina in vivo imaging system confirmed the in vivo results with a higher accumulation of anti-GPC1 C in the tumor mass compared to the IgM pool (FIGS. 5E and 5F).

Ex vivo assessment at 96 hours by immunofluorescence analysis still detected the anti-GPC1-C antibody in the BXPC3 tumor mass, confirming the ability of the anti-GPC1-C antibody to reach the tumor mass and remain in the tumor for a relevant period of time (at least 96 hours) (FIG. 5G).

Ex vivo evaluation of tumor masses of mice injected with the murine IgM pool or the PBS solution confirmed a specific anti-GPC1 driven capability of the anti-GPC1 C antibody of reaching GPC1 expressing BXPC3 tumor mass, as the signal was absent with the two controls (FIG. 5G).

### Example 4

### Anti-GPC1 C is Able to Induce Complement Dependent Cytotoxicity In Vivo

Anti-GPC1 C was shown to induce complement-dependent cytotoxicity (CDC) in BXPC3 tumor mass in xenograft mouse models obtained by subcutaneous injection of BXPC3 cells into athymic nude mice. Ex vivo assessment of tumor masses of BXPC3 xenograft mice by hematoxylin-eosin staining after 96 hours revealed a strong purple staining (hematoxylin) associated with a high content of nucleic acids within the tumor. This is a clear indicator of cell death in the mice treated with the anti-GPC1 C compared with those treated with the murine IgM pool or with the PBS solution (FIG. 6).

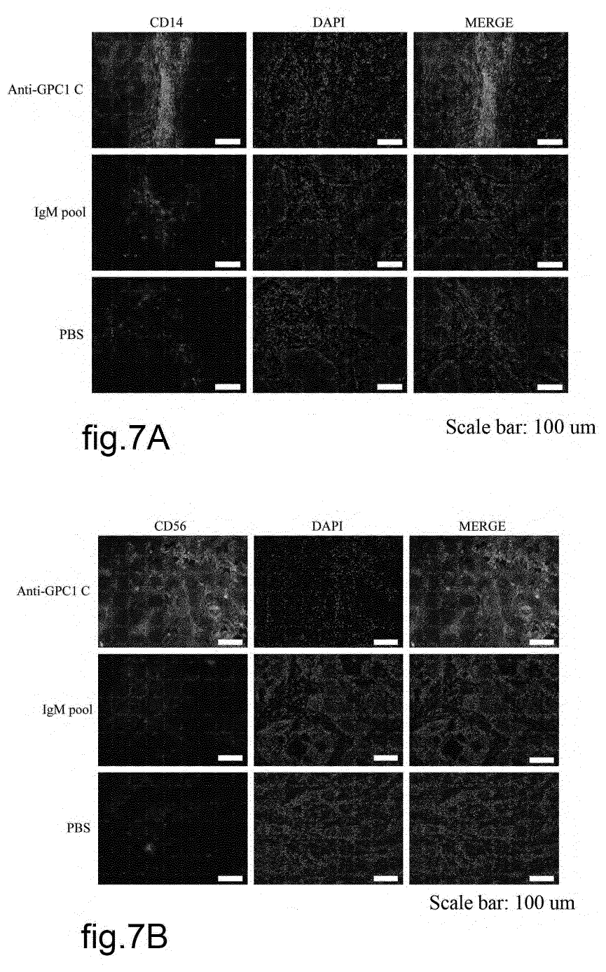

To better characterize the source of cell death, the presence of complement system effectors, but also the recruitment of macrophages, and NK cells was also analyzed by immunofluorescence analysis (FIGS. 7A, 7B).

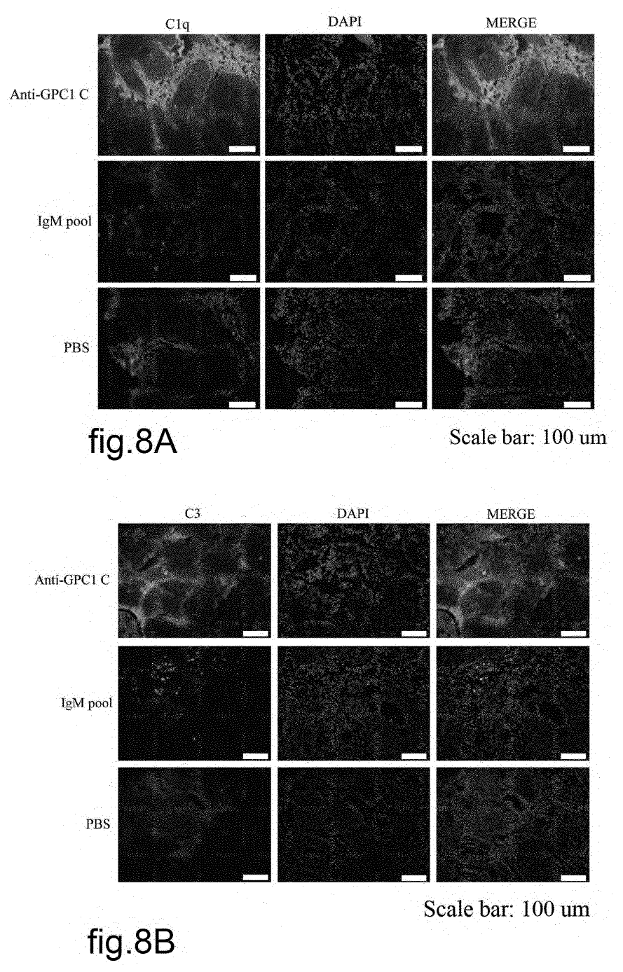

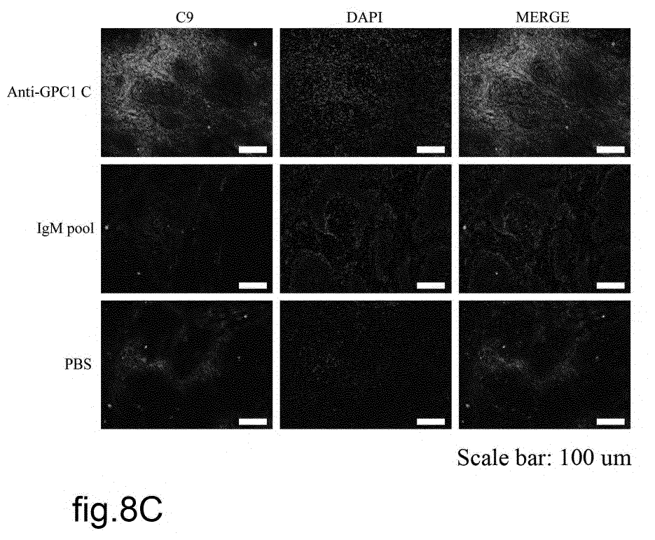

In particular, the involvement of the complement system was investigated, by analyzing C1q, C3 (C3b, iC3b, C3c), and C9. The results showed a high degree of complement system deposition (C1q, C3, C9), macrophage and NK cell recruitment in the BXPC3 tumors of mice treated with the anti-GPC1 C compared with the murine IgM pool or PBS solution (FIGS. 8A, 8B, 8C).

### Example 5

### Anti-GPC1 C Reduces GPC1-Expressing Tumor Masses In Vivo

The anti-GPC1 C was shown to reduce tumor growth when used to treat BXPC3 injected xenograft mouse models. This evidence has been demonstrated in an in vivo experiment in which 2 groups of 7 mice, in which the GPC1-expressing BXPC3 model was established, were employed. One group was intra-peritoneally treated with 38 micrograms of the anti-GPC1 C twice a week, whereas the other group was treated twice a week with PBS as a control. The treatment procedure started when the tumor reached a volume of 75 mm³, and when possible, it was stopped at day 42. For mice reaching the day 42, the final experimental endpoint was set up at day 50 from the beginning of the treatment procedure. Alternatively, mice were treated until reaching the humanitarian endpoint (one dimension of the tumor greater than 12 mm) or ulceration of the tumor. In this experiment, mice treated with the anti-GPC1 C showed a mean day of euthanasia or sacrifice of 42.7 (e.g., 28, 29, 42, 50, 50, 50, 50) with the 57.1% of animals reaching the pre-established experimental endpoint of 50 days. Mice treated with PBS showed a mean day of euthanasia of 13.7 (e.g., 10, 10, 14, 14, 15, 15, 18) (Table 1).

TABLE 1
Anti-GPC1 C-based immunotherapy study summary

| | | | Day | | % |
|---|---|---|---|---|---|
| | | | of | Mean day of | survival |
| | Endpoint | Endpoint | end | euthanasia/ | at day |
| Mouse | reached | typology | point | sacrifice | 50 |
| Untreated | humanitarian | dimension | 10 | 13.7 | 0 |
| Untreated | humanitarian | dimension | 10 | | |
| Untreated | humanitarian | dimension | 14 | | |
| Untreated | humanitarian | dimension | 14 | | |
| Untreated | humanitarian | dimension | 18 | | |
| Untreated | humanitarian | tumor | 14 | | |
| | | ulceration | | | |
| Untreated | humanitarian | tumor | 15 | | |
| | | ulceration | | | |
| Treated | humanitarian | dimension | 28 | 42.7 | 57.1 |
| Treated | humanitarian | dimension | 29 | | |
| Treated | humanitarian | dimension | 42 | | |
| Treated | experimental | / | 50 | | |
| Treated | experimental | / | 50 | | |
| Treated | Experimental | / | 50 | | |
| Treated | experimental | / | 50 | | |

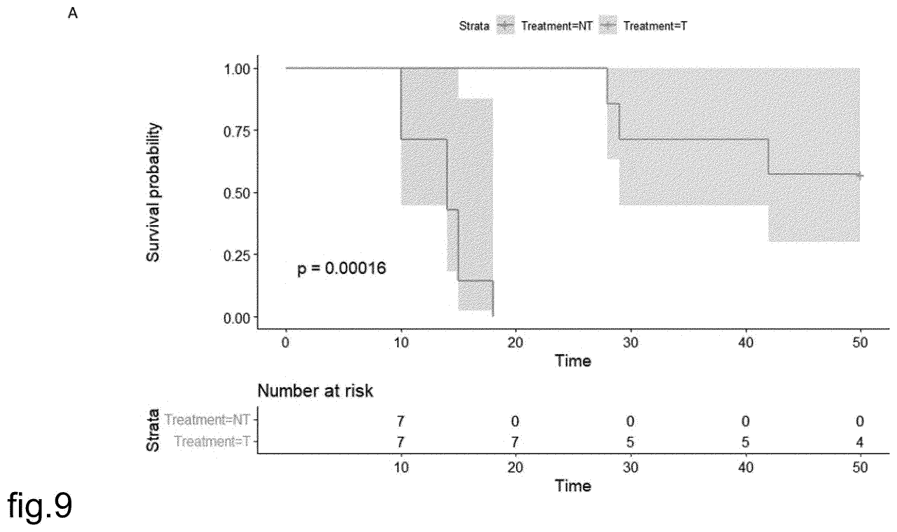

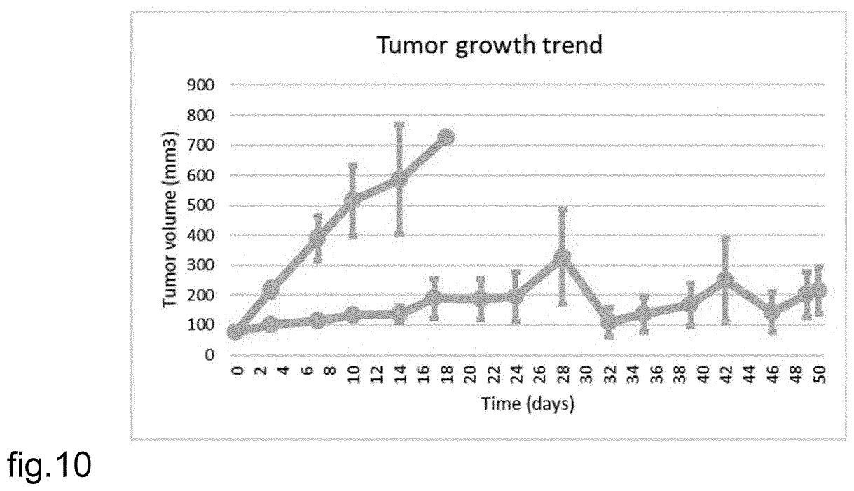

Survival time intervals of mice treated with the anti-GPC1 C were significantly longer than those of mice treated with PBS (p=0.00016, log-rank test, FIG. 9, FIG. 10).

In this case, the humanitarian endpoints causing the euthanasia of mice belonging to the group treated with PBS were for the 71.4% of mice (5 mice) a dimension greater than 12 mm and for the 28.6% of the mice (2 mice) the ulceration of the tumor. In the group of mice treated with the anti-GPC1 C, euthanasia was applied for 3 mice having a dimension of the tumor greater than 12 mm. Of note, from the day 19 to the day 27, the total of mice belonging to the group treated with PBS were all euthanized, whereas the mice belonging to the group treated with the anti-GPC1 C were all still alive. At the experimental endpoint at day 50, the percentage of survival for the group treated with the anti-GPC1 C was 57.1%. In addition, in one animal the complete remission of the tumor mass was observed. In the group of mice treated with the anti-GPC1 C, no evidence of toxicity (e.g., weight loss, diarrhea, vomiting, convulsions, dehydration, tachypnea, dyspnea, motionless) was observed.

### Example 6

Anti-GPC1 C Specifically Targets Polymeric Nanoparticles In Vivo in the BXPC3 Xenograft mouse model

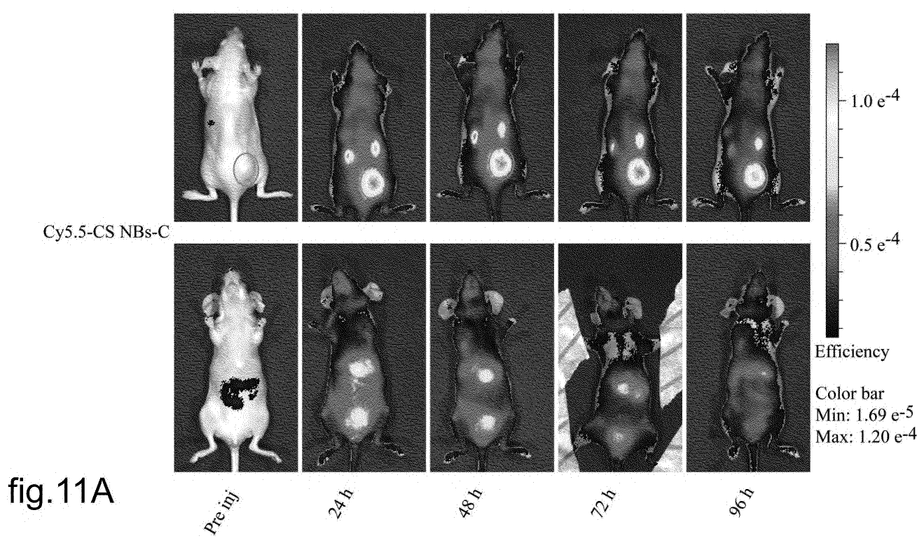

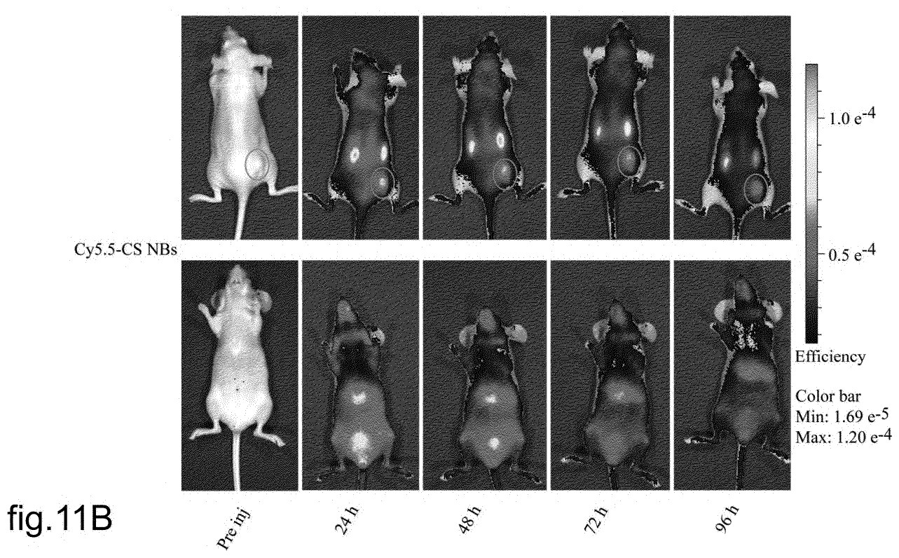

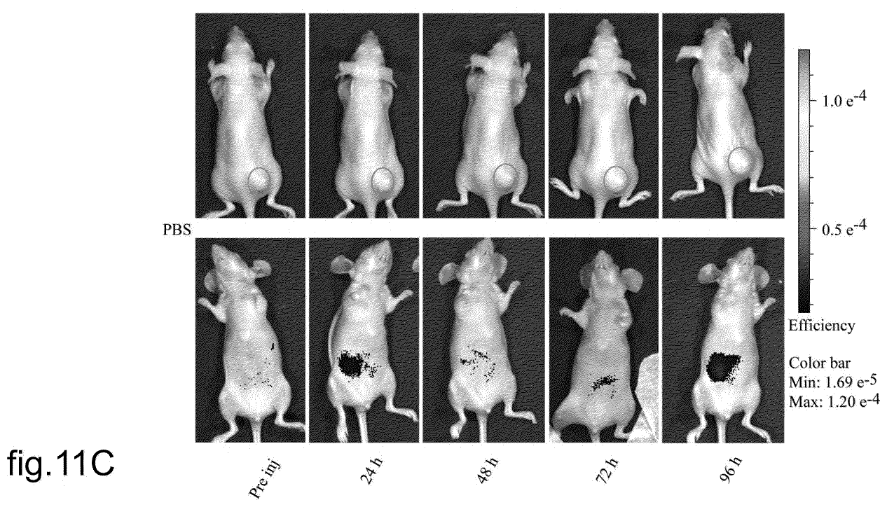

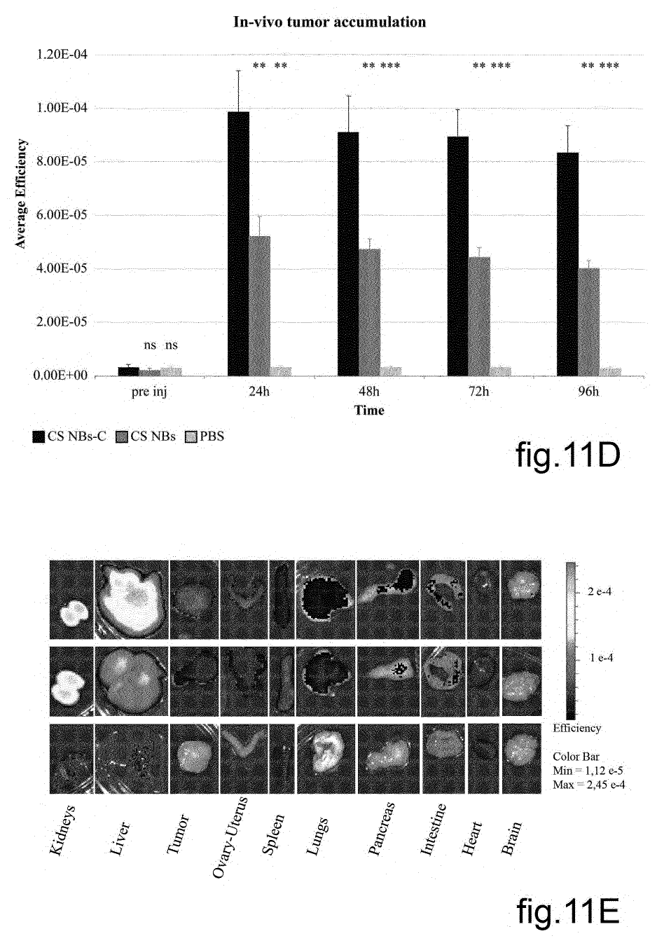

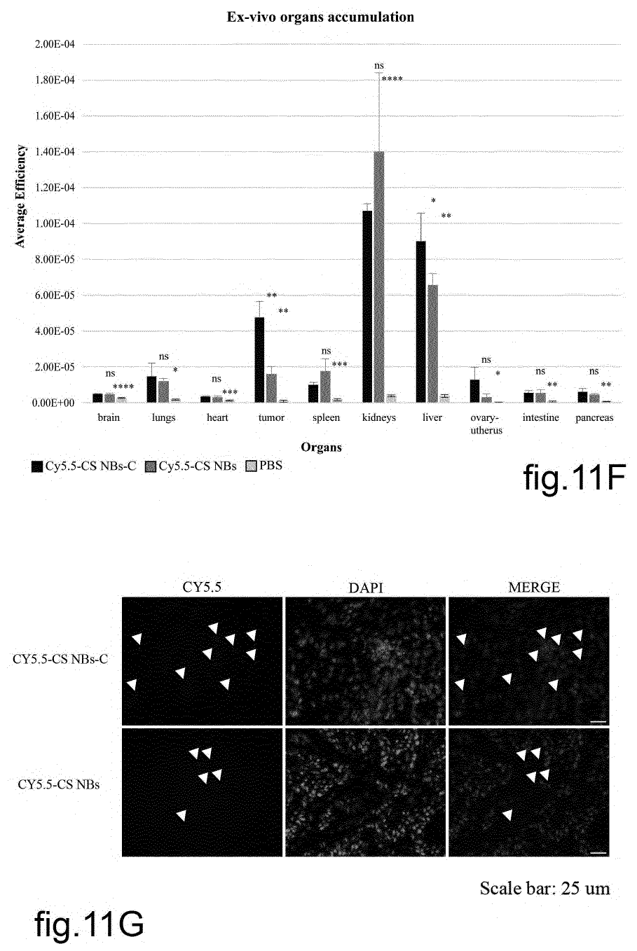

The anti-GPC1 C was shown to increase the capability of polymeric nanoparticles (e.g., chitosan nanobubbles) to reach the BXPC3 tumor mass in xenograft mouse models obtained by subcutaneously injecting the BXPC3 cells in athymic nude mice. In detail, in vivo time course experiments with a signal detection from 0 to 96 hours and a signal evaluation every 24 hours were performed to evaluate the capability to reach the tumor mass and the overall biodistribution of chitosan nanobubbles conjugated with the anti-GPC1 C (CS NBs C) compared to chitosan nanobubbles without antibody conjugation (CS NBs). Both the CS NBs preparations were injected intra-vein in the mouse tail after conjugation with Cy5.5 in 2 different groups of 4 mice to allow detection by using the In vivo Imaging VIVOVISION IVIS®Lumina (Xenogen) instrument. A third comparison group of 4 mice was used as a control group by injecting PBS solution. An amount of conjugated CS NBs corresponding to 1 nmol of Cy5.5 was injected into the tail vain of the mice for both the preparation of NBs. As reported in the representative distributions in the whole mouse bodies in both the supine and prone positions, the main hotspots of accumulation were the tumor masses and the kidneys (FIGS. 11A, 11B and 11C).

Moreover, results of the experiments showed that the conjugation with the anti-GPC1 C significantly increased the amount of CS NBs reaching the tumor masses at each evaluated time points (p=0.0043 at 24 hours, p=0.0054 at 48 hours, p=0.0013 at 72 hours, p=0.002 at 96 hours), and the peak of accumulation was reached at 24 hours (FIG. 11D). As expected, the group of mice treated with PBS did not show any signal over time.

The data obtained in vivo were confirmed ex vivo by In vivo Imaging VIVOVISION IVIS®Lumina reconfirming the importance of the anti-GPC1 C as agent of active targeting (FIGS. 11E and 11F).

The preferential localization of CS NBs C within the tumor was further confirmed by IF analyzing cy5.5, FIG. 11G clearly highlighted the high quantity of CS NBs-C that had reached the tumor site.

### Example 7

### Anti-GPC1 C Increases the Efficacy of Polymeric Nanoparticles Treatment In Vivo Through Specific Active Targeting in the BXPC3 Mouse Model

The anti-GPC1 C was shown to increase the efficacy of the treatment of polymeric nanoparticles (chitosan nanobubbles) loaded with doxorubicin in reducing BXPC3 tumor masses in vivo. Both the doxorubicin-loaded chitosan nanobubbles conjugated with the anti-GPC1 C (doxo-CS NBs-C) and without anti-GPC1 C conjugation (doxo-CS NBs) were injected intra-peritoneum of the mice in a dosage corresponding to 2 mg/kg per week, subdivided in three different administrations. The study started when the subcutaneous BXPC3-tumor reached a volume of 75 mm³. The humanitarian endpoints that had caused the euthanasia of mice were: one tumor dimension greater than 12 mm or ulceration of the tumor. Four groups of mice were tested: a first group of animals treated with doxo-CS NBs-C (n=6); a second group of animals treated with doxo-CS NBs (n=6); a third group of control animals treated with doxorubicin (doxo 2 mg/kg) in order to treat mice at the same concentration (2 mg/kg for a week) (n=10); a fourth group of control animals treated with PBS solution (n=8). Interestingly, the 94% of the animals treated with doxo-CS NBs-C reached the pre-established experimental endpoint of 29 days, showing a promising antitumor activity. Furthermore, in one mouse complete remission of the tumor was observed (Table 2).

TABLE 2
Mean survival day of the experimental groups

| Experimental group | Mean survival ± S.D. (days) |
|---|---|
| Untreated | 10.12 ± 1.96 |
| Doxo 2 mg/kg | 13.9 ± 1.59 |
| Doxo-CS NBs | 19 ± 4.56 |
| Doxo-CS NBs-C | 27.5 ± 3.67 |

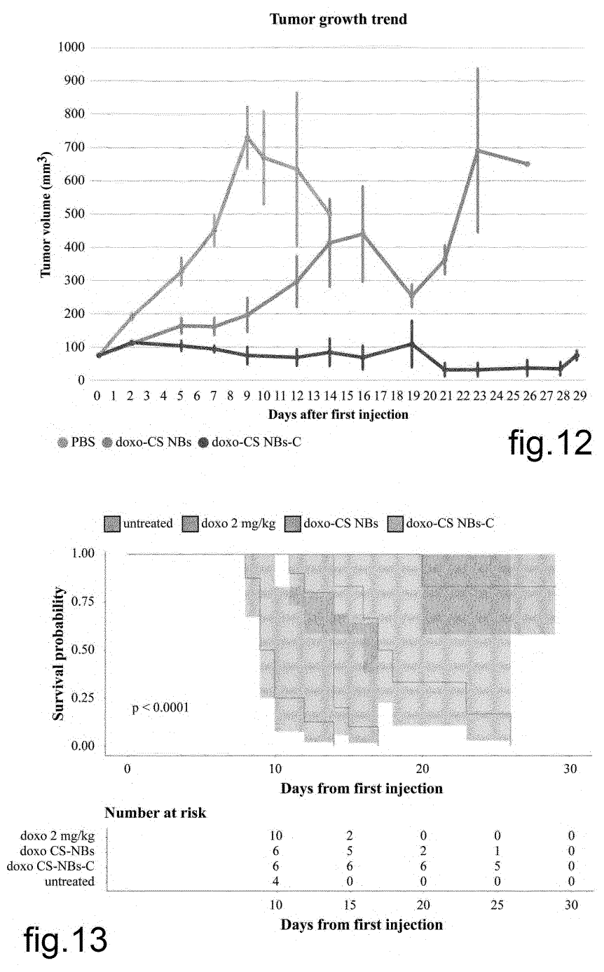

On the other hand, none of the animals treated with doxo-CS NBs reached the pre-established experimental endpoint of 29 days, thus confirming the capability of the anti-GPC1 C to increase efficacy of doxorubicin-loaded chitosan nanobubbles through active targeting. Mice survival was evaluated using Kaplan-Meier curves and the p-value was calculate using log rank test. Both the preparations of CS NBs significantly increased the survival of the mice if compared with untreated group and with the doxo 2 mg/kg per week regimen; the global Kaplan-Meier curve showed a p-value <0.0001 calculated using log rank test (FIG. 12 and FIG. 13).

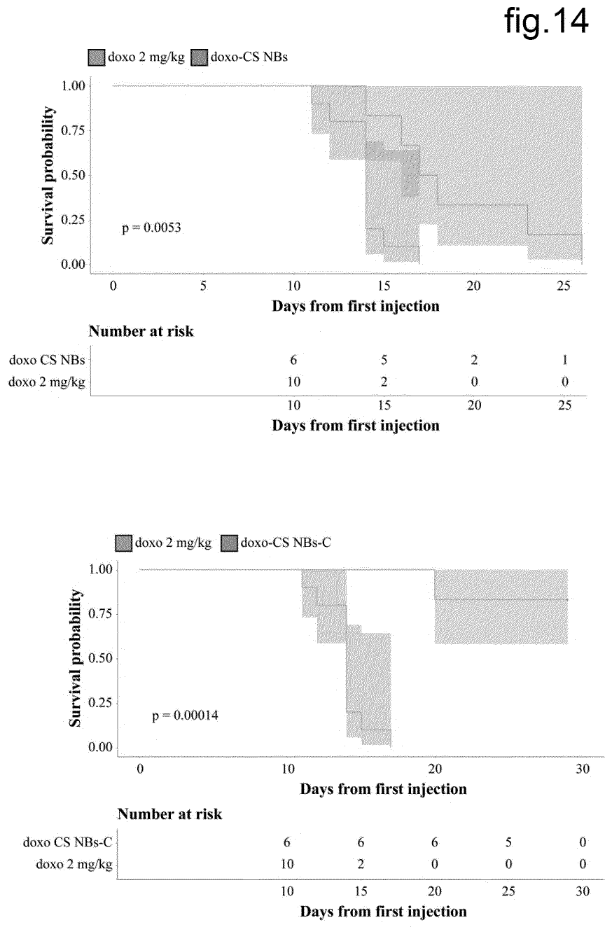

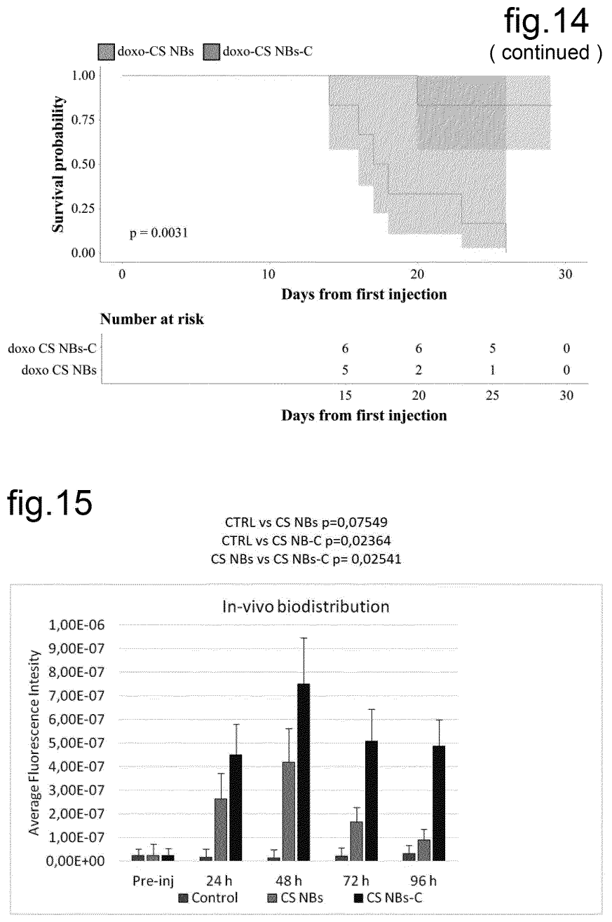

Each treatment, compared with the untreated group, increased the survival of the animals. Furthermore, the employment of both CS NBs preparations increased the survival of the mice if compared with the regimen of doxo 2 mg/kg per week; doxo-CS NBs showed a p-value of 0.0053, while doxo-CS NBs-C showed a p-value of 0.00014. The comparison between doxo-CS NBs and doxo-CS NBs showed a favourable prognosis in favour of doxo-CS NBs-C, p-value of 0.0031 (FIG. 14).

The evaluation of HR demonstrated that each treatment was protective compared with untreated group: doxo 2 mg/Kg per week 0.19 (p-value of 0.002); doxo-CS NBs 0.03 (p-value <0.001); doxo-CS NBs-C 0.00 (p-value <0.001) (Table 3). Doxo-CS NBs 2 mg/kg per week and doxo-CS NBs-C 2 mg/kg per week showed to be protective in comparison with the same dosage of free doxo; in detail, doxo-CS NBs exhibited an HR of 0.16 (p-value of 0.010) while doxo-CS NBs-C exhibited an HR of 0.01 (p-value <0.001) (Table 3). As expected, based on FIG. 6C, the doxo-CS NBs-C improved the prognosis of the mice if compared with the group treated with doxo-CS NBs, with an HR of 0.07 (p-value of 0.018) (Table 3).

TABLE 3
HR and the p-values of the experimental groups
Comparison treatment

| | | | | Doxo-CS NBs | | Doxo-CS NBs-C | |
|---|---|---|---|---|---|---|---|
| | | Doxo 2 mg/kg | | 2 mg/kg | | 2 mg/kg | |
| | | HR (95% | | HR (95% | | HR (95% | |
| | | confidence | P | confidence | P | confidence | P |
| | | interval) | value | interval) | value | interval) | value |
| Reference | Untreated | 0.19 | 0.002 | 0.03 | <0.001 | 0.00 | <0.001 |
| treatment | | (0.07-0.55) | | (0.01-0.15) | | (0.00-0.03) | |
| | Doxo 2 | | | 0.16 | 0.010 | 0.01 | <0.001 |
| | mg/kg | | | (0.04-0.64) | | (0.00-0.13) | |
| | Doxo-CS | | | | | 0.07 | 0.018 |
| | NBs | | | | | (0.01-0.64) | |

### Example 8

### Anti-GPC1 C Specifically Targets Polymeric Nanoparticles In Vivo in the U87MG Xenograft Mouse Model

The anti-GPC1 C was shown to increase the capability of polymeric nanoparticles (e.g., chitosan nanobubbles) to reach the U87MG tumor mass in xenograft mouse models obtained by subcutaneously injecting the U87MG cells in athymic nude mice. In detail, in vivo time course experiments with a signal detection from 0 to 96 hours and a signal evaluation every 24 hours were performed to evaluate the capability to reach the tumor mass and the overall biodistribution of chitosan nanobubbles conjugated with the anti-GPC1 C (CS NBs C) compared to chitosan nanobubbles without antibody conjugation (CS NBs). Both the CS NBs preparations were injected intra-vein in the mouse tail after conjugation with Cy5.5 in 2 different groups of 4 mice to allow detection by using the In vivo Imaging VIVOVISION IVIS®Lumina (Xenogen) instrument. A third comparison group of 4 mice was used as control group by injecting PBS solution. An amount of conjugated CS NBs corresponding to 1 nmol of Cy5.5 were injected into the tail vain of the mice for both preparations of NBs.

After the treatments, mice were followed at different time points (24, 48, 72, 96 h). At 48 hours after treatment with NBs (CS NB, CS NB C) a peak of NBs accumulation in the U87MG tumor mass was shown both in the CS NB C group and in the CS NBs group. By considering the Cy5.5 fluorescence intensity, CS NB C group yield improved signal-to-background ratios in comparison to CS NB group (p=0.02541) within the tumor site, considering the entire series of fluorescence intensity values from 24 hours until 96 hours after injection (FIG. 15). Moreover, it is notable that the retention time of CS NB C was higher than CS NB when tested in mice sacrificed at the 96 h time point although without reaching a significant difference. Overall considered, these results showed that conjugation with the C antibody allows a major accumulation of the injected NBs in the tumor as well as a higher retention time at least until the last time point of 96 h of treatment.

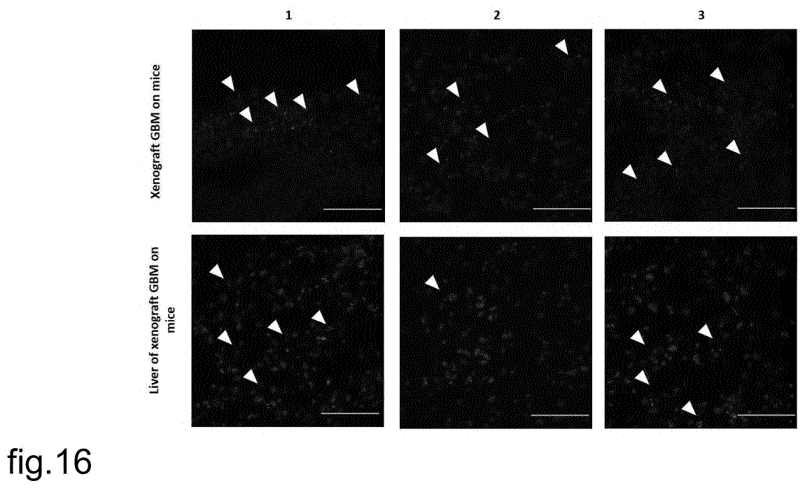

The selective localization of CS NB C in tumor masses and in the liver was further confirmed by evaluating the presence the CS NB C using fluorescent microscopy. In keeping with the IVIS images, the analyses of U87MG tumor tissue and liver cryosections isolated from mice sacrificed at 96 hours revealed a non-homogeneously distributed fluorescence signal, consistent with the presence of the CS NB C (FIG. 16). These results confirmed the accumulation of CS NB C in the tumor and the liver.

### REFERENCES

1 Li, N., Gao, W., Zhang, Y. F. & Ho, M. Glypicans as Cancer Therapeutic Targets. Trends Cancer 4, 741-754, doi: 10.1016/j.trecan.2018.09.004 (2018).
2 Hara, H. et al. Overexpression of glypican-1 implicates poor prognosis and their chemoresistance in oesophageal squamous cell carcinoma. Br J Cancer 115, 66-75, doi: 10.1038/bjc.2016.183 (2016).
3 Kleeff, J. et al. The cell-surface heparan sulfate proteoglycan glypican-1 regulates growth factor action in pancreatic carcinoma cells and is overexpressed in human pancreatic cancer. J Clin Invest 102, 1662-1673, doi: 10.1172/JCI4105 (1998).
4 Li, J. et al. Glypican-1 Promotes Tumorigenesis by Regulating the PTEN/Akt/beta-Catenin Signaling Pathway in Esophageal Squamous Cell Carcinoma. Dig Dis Sci 64, 1493-1502, doi: 10.1007/s10620-019-5461-9 (2019).
5 Lu, H. et al. Elevated glypican-1 expression is associated with an unfavorable prognosis in pancreatic ductal adenocarcinoma. Cancer Med 6, 1181-1191, doi: 10.1002/cam4.1064 (2017).
6 Melo, S. A. et al. Glypican-1 identifies cancer exosomes and detects early pancreatic cancer. Nature 523, 177-182, doi: 10.1038/nature14581 (2015).
7 Quach, N. D. et al. Paradoxical Role of Glypican-1 in Prostate Cancer Cell and Tumor Growth. Sci Rep 9, 11478, doi: 10.1038/s41598-019-47874-2 (2019).
8 Saito, T. et al. High Expression of Glypican-1 Predicts Dissemination and Poor Prognosis in Glioblastomas. World Neurosurg 105, 282-288, doi: 10.1016/j.wneu.2017.05.165 (2017).
9 Su, G. et al. Glypican-1 is frequently overexpressed in human gliomas and enhances FGF-2 signaling in glioma cells. Am J Pathol 168, 2014-2026, doi: 10.2353/ajpath.2006.050800 (2006).
10 Zhou, C. Y. et al. High levels of serum glypican-1 indicate poor prognosis in pancreatic ductal adenocarcinoma. Cancer Med 7, 5525-5533, doi: 10.1002/cam4.1833 (2018).
11 Cruz, E. & Kayser, V. Monoclonal antibody therapy of solid tumors: clinical limitations and novel strategies to enhance treatment efficacy. Biologics 13, 33-51, doi: 10.2147/BTT.S166310 (2019).
12 Dong, S. & Ghobrial, I. M. Immunotherapy for hematological malignancies. J Life Sci (Westlake Village) 1, 46-52 (2019).
13 Hester, R., Mazur, P. K. & McAllister, F. Immunotherapy in Pancreatic Adenocarcinoma: Beyond “Copy/Paste”. Clin Cancer Res 27, 6287-6297, doi: 10.1158/1078-0432.CCR-18-0900 (2021).
14 Shah, A. et al. The Current Landscape of Antibody-based Therapies in Solid Malignancies. Theranostics 11, 1493-1512, doi: 10.7150/thno.52614 (2021).
15 Burger, R. A. et al. Incorporation of bevacizumab in the primary treatment of ovarian cancer. N Engl J Med 365, 2473-2483, doi: 10.1056/NEJMoa1104390 (2011).
16 Burns, M. C., O'Donnell, A. & Puzanov, I. Pembrolizumab for the treatment of advanced melanoma. Expert Opin Orphan Drugs 4, 867-873, doi: 10.1080/21678707.2016.1191348 (2016).
17 Gettinger, S. et al. Nivolumab Monotherapy for First-Line Treatment of Advanced Non-Small-Cell Lung Cancer. J Clin Oncol 34, 2980-2987, doi: 10.1200/JCO.2016.66.9929 (2016).
18 Kansara, R. R. & Speziali, C. Immunotherapy in hematologic malignancies. Curr Oncol 27, S124-S131, doi: 10.3747/co.27.5117 (2020).
19 Slamon, D. et al. Adjuvant trastuzumab in HER2-positive breast cancer. N Engl J Med 365, 1273-1283, doi: 10.1056/NEJMoa0910383 (2011).
20 Schroeder, H. W., Jr. & Cavacini, L. Structure and function of immunoglobulins. J Allergy Clin Immunol 125, S41-52, doi: 10.1016/j.jaci.2009.09.046 (2010).
21 Lambert, J. M. & Chari, R. V. Ado-trastuzumab Emtansine (T-DM1): an antibody-drug conjugate (ADC) for HER2-positive breast cancer. J Med Chem 57, 6949-6964, doi: 10.1021/jm500766w (2014).
22 Senter, P. D. & Sievers, E. L. The discovery and development of brentuximab vedotin for use in relapsed Hodgkin lymphoma and systemic anaplastic large cell lymphoma. Nat Biotechnol 30, 631-637, doi: 10.1038/nbt.2289 (2012).
23 Tedeschini, T. et al. Polyethylene glycol-based linkers as hydrophilicity reservoir for antibody-drug conjugates. J Control Release 337, 431-447, doi: 10.1016/j.jconrel.2021.07.041 (2021).
24 Navya, P. N. et al. Current trends and challenges in cancer management and therapy using designer nanomaterials. Nano Converg 6, 23, doi: 10.1186/s40580-019-0193-2 (2019).
25 Chaturvedi, V. K., Singh, A., Singh, V. K. & Singh, M. P. Cancer Nanotechnology: A New Revolution for Cancer Diagnosis and Therapy. Curr Drug Metab 20, 416-429, doi: 10.2174/1389200219666180918111528 (2019).
26 Zhao, C. Y., Cheng, R., Yang, Z. & Tian, Z. M. Nanotechnology for Cancer Therapy Based on Chemotherapy. Molecules 23, doi: 10.3390/molecules23040826 (2018).
27 Banik, B. L., Fattahi, P. & Brown, J. L. Polymeric nanoparticles: the future of nanomedicine. Wiley Interdiscip Rev Nanomed Nanobiotechnol 8, 271-299, doi: 10.1002/wnan.1364 (2016).
28 Prabhu, R. H., Patravale, V. B. & Joshi, M. D. Polymeric nanoparticles for targeted treatment in oncology: current insights. Int J Nanomedicine 10, 1001-1018, doi: 10.2147/IJN.S56932 (2015).
29 Cavalli, R. et al. New chitosan nanobubbles for ultrasound-mediated gene delivery: preparation and in vitro characterization. Int J Nanomedicine 7, 3309-3318, doi: 10.2147/IJN.S30912 (2012).
30 Danhier, F. et al. PLGA-based nanoparticles: an overview of biomedical applications. J Control Release 161, 505-522, doi: 10.1016/j.jconrel.2012.01.043 (2012).
31 Rizeq, B. R., Younes, N. N., Rasool, K. & Nasrallah, G. K. Synthesis, Bioapplications, and Toxicity Evaluation of Chitosan-Based Nanoparticles. Int J Mol Sci 20, doi: 10.3390/ijms20225776 (2019).
32 Kang, H. et al. Size-Dependent EPR Effect of Polymeric Nanoparticles on Tumor Targeting. Adv Healthc Mater 9, e1901223, doi: 10.1002/adhm.201901223 (2020).
33 Singh, R. R. & O'Reilly, E. M. New Treatment Strategies for Metastatic Pancreatic Ductal Adenocarcinoma. Drugs 80, 647-669, doi: 10.1007/s40265-020-01304-0 (2020).
34 Stupp, R. et al. Radiotherapy plus concomitant and adjuvant temozolomide for glioblastoma. N Engl J Med 352, 987-996, doi: 10.1056/NEJMoa043330 (2005).
35 Lu, R. M. et al. Development of therapeutic antibodies for the treatment of diseases. J Biomed Sci 27, 1, doi: 10.1186/s12929-019-0592-z (2020).
36 Fus-Kujawa, A. et al. An Overview of Methods and Tools for Transfection of Eukaryotic Cells in vitro. Front Bioeng Biotechnol 9, 701031, doi: 10.3389/fbioe.2021.701031 (2021).
37 Kuriu, Y. et al. Monoclonal antibody conjugated to gadolinium as a contrast agent for magnetic resonance imaging of human rectal carcinoma. J Surg Oncol 94, 144-148, doi: 10.1002/jso.20411 (2006).

1. A monoclonal antibody binding to the antigen glypican-1 (GPC1), wherein the monoclonal antibody is of IgM isotype and comprises three heavy chain complementarity determining regions HCDR1, HCDR2, and HCDR3 and three light chain complementarity determining regions LCDR1, LCDR2, and LCDR3, wherein:
HCDR1 includes the amino acid sequence of SEQ ID NO: 3,
HCDR2 includes the amino acid sequence of SEQ ID NO: 5,
HCDR3 includes the amino acid sequence of SEQ ID NO: 7,
LCDR1 includes the amino acid sequence of SEQ ID NO: 9,
LCDR2 includes the amino acid sequence of KVS, and
LCDR3 includes the amino acid sequence of SEQ ID NO: 12.

2. The monoclonal antibody of claim 1, comprising a heavy chain variable region including the amino acid sequence of SEQ ID NO: 13 and a light chain variable region including the amino acid sequence of SEQ ID NO: 14.

3. The monoclonal antibody of claim 1, wherein the monoclonal antibody is a pentameric IgM.

4. The monoclonal antibody of claim 1, wherein the monoclonal antibody is a monomeric IgM.

5. The monoclonal antibody of claim 1, which wherein the monoclonal antibody is conjugated with an antitumor drug or with a polymeric nanoparticle loaded with an antitumor drug.

6. (canceled)

7. (canceled)

8. A nucleic acid expression construct encoding the monoclonal antibody of claim 1.

9. A host cell transformed with the nucleic acid expression construct of claim 8.

10. A pharmaceutical preparation comprising the monoclonal antibody of claim 1 and at least one pharmaceutically acceptable excipient, vehicle or diluent.

11. A method of inducing complement-dependent cytotoxicity and/or reducing a tumor mass in a subject affected by a GPC1-expressing tumor, the method comprising administering to the subject the monoclonal antibody of claim 1.

12. A method of recombinantly producing the monoclonal antibody of claim 1, the method comprising cultivating a host cell transformed with a nucleic acid expression construct encoding the monoclonal antibody under conditions suitable for the expression of the nucleic acid expression construct, and recovering the expressed monoclonal antibody.

13. The method of claim 12, wherein the recovered monoclonal antibody is further conjugated with an antitumor drug or with a polymeric nanoparticle loaded with an antitumor drug.

14. An in vitro method of detecting a GPC1-expressing tumor, comprising contacting a sample from a subject suspected of being affected by the GPC1-expressing tumor with a detection reagent comprising the monoclonal antibody of claim 1, and detecting binding of the detection reagent with the sample, wherein the binding is indicative of the GPC1-expressing tumor.

15. The in vitro method of claim 14, wherein the sample is a cell sample or a tissue sample.

16. A nucleic acid expression construct encoding the monoclonal antibody of claim 2.

17. A host cell transformed with the nucleic acid expression construct of claim 16.

18. A pharmaceutical preparation comprising the monoclonal antibody of claim 2 and at least one pharmaceutically acceptable excipient, vehicle or diluent.

19. The method of claim 11, wherein the GPC1-expressing tumor is selected from the group consisting of pancreatic adenocarcinoma, glioblastoma, prostate cancer, and squamous cell carcinoma of the esophagus.

20. A method of inducing complement-dependent cytotoxicity and/or reducing a tumor mass in a subject affected by a GPC1-expressing tumor, the method comprising administering to the subject the monoclonal antibody of claim 2.

21. The method of claim 20, wherein the GPC1-expressing tumor is selected from the group consisting of pancreatic adenocarcinoma, glioblastoma, prostate cancer, and squamous cell carcinoma of the esophagus.

22. An in vitro method of detecting a GPC1-expressing tumor, comprising contacting a sample from a subject suspected of being affected by the GPC1-expressing tumor with a detection reagent comprising the monoclonal antibody of claim 2, and detecting binding of the detection reagent with the sample, wherein the binding is indicative of the GPC1-expressing tumor.


SEQUENCE LISTING (14 sequences)

SEQ ID NO: 1 - protein, 70 aa, Homo sapiens
GSGDGCLDDLCSRKVSRKSSSSRTPLTHALPGLSEQEGQKTSAASCPQPPTFLLPLLLFLALTVARPRWR

SEQ ID NO: 2 - protein, 25 aa, Mus musculus
QVQLQQSDAELVKPGASVKISCKAS

SEQ ID NO: 3 - protein, 8 aa, Mus musculus
GYTFTDHA

SEQ ID NO: 4 - protein, 17 aa, Mus musculus
IHWVKQKPEQGLEWIGY

SEQ ID NO: 5 - protein, 8 aa, Mus musculus
ISPGNGDI

SEQ ID NO: 6 - protein, 38 aa, Mus musculus
KYNEKFKGKATLTADKSSSTAYMQLNSLTSEDSAVYFC

SEQ ID NO: 7 - protein, 16 aa, Mus musculus
KRYAYWGQGTLVTVSA

SEQ ID NO: 8 - protein, 26 aa, Mus musculus
DVLMTQTPLSLPVSLGDQASISCRSS

SEQ ID NO: 9 - protein, 11 aa, Mus musculus
QSIVHSNGNTY

SEQ ID NO: 10 - protein, 17 aa, Mus musculus
LEWYLQKPGQSPKLLIY

SEQ ID NO: 11 - skipped

SEQ ID NO: 12 - protein, 20 aa, Mus musculus
FQGSHVPWTFGGGTKLEIKP

SEQ ID NO: 13 - protein, 112 aa, Mus musculus
QVQLQQSDAELVKPGASVKISCKASGYTFTDHAIHWVKQKPEQGLEWIGYISPGNGDIKYNEKFKGKATLTADKSSSTAYMQLNSLTSEDSAVYFCKRYAYWGQGTLVTVSA

SEQ ID NO: 14 - protein, 113 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIKP